(19) United States
(12) Patent Application Publication
(10) Pub. No.: US 2025/0334558 A1
(43) Pub. Date: Oct. 30, 2025

(54) TOTAL HYDROGEN SULFIDE QUANTIFICATION

(71) Applicant: The Cleveland Clinic Foundation, Cleveland, OH (US)

(72) Inventor: Stanley Hazen, Cleveland, OH (US)

(21) Appl. No.: 18/880,122

(22) PCT Filed: Jun. 29, 2023
(86) PCT No.: PCT/US2023/069400

§ 371 Date: Dec. 30, 2024

Related U.S. Application Data
(60) Provisional application No. 63/357,349, filed on Jun. 30, 2022.

(51) Int. Cl. G01N 33/00; G01N 30/06; G01N 30/88

(52) U.S. Cl. CPC G01N 33/0044; G01N 30/06; G01N 30/88; G01N 2030/8868

(57) ABSTRACT

The present invention relates to methods, compositions, systems, and kits for the analysis of hydrogen sulfide using an ³⁴S isotope-labeled sulfide compound (e.g., ³⁴S isotope-labeled sodium sulfide), a reducing agent and derivatization reagent, in particular by isotope dilution mass spectrometry.

### CROSS-REFERENCE TO RELATED APPLICATIONS

This application claims priority to and the benefit of U.S. Provisional Patent Application No. 63/357,349, filed on Jun. 30, 2022, which is incorporated herein by reference in its entirety.

### FIELD

The present invention relates to methods, compositions, systems, and kits for the analysis of hydrogen sulfide using an ³⁴S isotope-labeled sulfide compound (e.g., ³⁴S isotope-labeled sodium sulfide), in particular by isotope dilution mass spectrometry.

### BACKGROUND

Hydrogen sulfide (H₂S) is an endogenously produced gaseous signaling molecule that plays important physiological roles in human health. In mammals, three distinct enzymes are known to produce H₂S: cystathionine γ-lyase (CSE), cystathionine β-synthase (CBS), and 3-mercaptopyruvate sulfur transferase (3-MST). H₂S can also be produced by enteric sulfur-reducing bacteria. Many studies have shown that H₂S possesses significant biological activities including a role in aging. For example, human studies have reported clinical associations between H₂S and aging, cardiovascular disease, and other degenerative diseases.

Despite the role of H₂S in numerous physiological processes and diseases, methods for measuring H₂S are limited by many factors including: difficulties in quantifying a volatile molecule; the presence of contaminating H₂S in commercial thiols and reducing agents; the need for specialized equipment; and the lack of an internal standard. Tissue generation of H₂S ex vivo is often quantified by reacting head space gas above tissues incubated with media with lead acetate, and following the color change observed with conversion to lead sulfide. Such methods do not enable quantification of in vivo H₂S levels at the time of sample collection, but instead measure subsequent synthetic enzyme capacity of the sample collected. Other methods for H₂S quantification involve derivatization with monobromobimane, fluorescent probes, or alternative reagents, and detection with either fluorescence or mass spectrometry. These methods, however, all rely on use of external calibration curves. Monobromobimane, one of the most commonly used derivatization reagents for H₂S quantification due to its florescence, has relatively poor ionization characteristics and is light sensitive, requiring that it be stored and reactions be performed in the dark and thereby limiting its practical use for the high throughput demands of an in vitro clinical diagnostic assay.

### SUMMARY

Provided herein are methods for detecting hydrogen sulfide in a sample using ³⁴S isotope-labeled sodium sulfide as a standard.

In some embodiments, the methods comprise adding a ³⁴S isotope-labeled sulfide compound (e.g., ³⁴S isotope-labeled sodium sulfide) and a reducing agent to the sample; incubating the sample with a derivatizing agent to form a derivatized sample; and analyzing the derivatized sample by isotope dilution mass spectrometry.

In some embodiments, the methods further comprise: detecting primary and secondary multiple reaction monitoring (MRM) transitions for ³⁴S isotope-labeled derivatized sulfide and unlabeled derivatized sulfide; calculating an area ratio of the primary unlabeled derivatized sulfide MRM transition to the ³⁴S isotope-labeled derivatized sulfide primary MRM transition; and determining a concentration of hydrogen sulfide by multiplying the concentration of added ³⁴S isotope-labeled sulfide with the area ratio.

In some embodiments, the ³⁴S isotope-labeled sulfide compound is ³⁴S isotope-labeled sodium sulfide. In some embodiments, the ³⁴S isotope-labeled sodium sulfide has an isotopic abundance of greater than 90% ³⁴S. In some embodiments, the ³⁴S isotope-labeled sodium sulfide is added in a basic buffer solution. In some embodiments, the basic buffer solution is ammonium bicarbonate at pH 10.

In some embodiments, the reducing agent is an agent capable of reducing polysulfides. In some embodiments, the reducing agent is tris(2-carboxyethyl) phosphine (TCEP). In some embodiments, the reducing agent is added to the sample at a final concentration of at least 1 mM. In some embodiments, the final concentration of the reducing agent is 1-100 mM.

In some embodiments, the ³⁴S isotope-labeled sulfide compound and the reducing agent are added simultaneously.

In some embodiments, the methods further comprise adding a chelating agent to the sample. In some embodiments, the chelating agent is diethylenetriaminepentaacetic acid (DTPA).

In some embodiments, the derivatizing agent is selected from ethyl iodoacetate, ethyl bromoacetate, methyl bromoacetate, and iodoacetate. In select embodiments, the derivatizing agent is ethyl iodoacetate. In some embodiments, the derivatizing agent is present in a molar excess compared to hydrogen sulfide in the sample. In some embodiments, the derivatizing agent is at a molar ratio of hydrogen sulfide to derivatizing agent of at least about 1:10. In some embodiments, the incubating is conducted for a time sufficient to allow detection of derivative products by mass spectrometry. In some embodiments, the derivatizing reaction is incubated for 8-24 hours. In some embodiments, the incubating is conducted at a temperature sufficient to allow detection of derivative products by mass spectrometry. In some embodiments, the derivatizing reaction is incubated at 20-23° C.

In some embodiments, the methods further comprise removing proteins in the derivatized sample.

In some embodiments, the isotope dilution mass spectrometry comprises liquid chromatography and tandem mass spectrometry.

In some embodiments, the primary MRM for unlabeled derivatized sulfide is 207.2→133.1 and the primary MRM for ³⁴S isotope-labeled derivatized sulfide is 209.2→135.1. In some embodiments, the secondary MRM for unlabeled derivatized sulfide is 207.2→105.0 and the secondary MRM for ³⁴S isotope-labeled derivatized sulfide is 209.2→107.1.

In some embodiments, the methods further comprise detecting additional thiol species in the sample by isotope dilution mass spectrometry. In some embodiments, the methods further comprise calculating the area ratio of the unlabeled derivatized sulfide secondary MRM transition to the ³⁴S isotope-labeled derivatized sulfide secondary MRM transition and comparing to area ratio of primary MRM transitions.

In some embodiments, the methods further comprise: adding an isotope labeled thiol compound to the sample. In some embodiments, the isotope labeled thiol compound is substantially free of H₂S. In some embodiments, the isotope labeled thiol compound comprises one or more or all of: [3,3-D₂] cysteine, [3,3,4,4-D₄] homocysteine, and (¹³C₂¹⁵N) glutathione.

In some embodiments, the methods further comprise: detecting primary and secondary MRM transitions for one or more or all of: cysteine, homocysteine, glutathione, cysteinylglycine and glutamylcysteine; calculating an area ratio of the primary MRM transition for one or more or all of: cysteine, homocysteine, glutathione, cysteinylglycine and glutamylcysteine to a corresponding isotope labeled thiol compound primary MRM transition; and determining a concentration by multiplying the concentration of corresponding isotope labeled thiol compound with the area ratio.

In some embodiments, the sample is a biological sample from a subject. In some embodiments, the biological sample is blood or a blood product.

In some embodiments, the methods further comprise predicting risk of a disease or disorder or death in a subject. In some embodiments, the methods further comprise comparing the hydrogen sulfide concentration from a subject sample to a control, wherein an increased hydrogen sulfide concentration indicates an increased risk of the disease or disorder. In some embodiments, the disease or disorder comprises cardiovascular disease. In some embodiments, the cardiovascular disease is selected from angina, arrhythmia, arteriosclerosis, atherosclerosis, myocardial infarction, acute coronary syndrome, cardiomyopathy, congestive heart failure, coronary thrombosis, aortic aneurysm, aortic dissection, iliac or femoral aneurysm, pulmonary embolism, high blood pressure/hypertension (e.g., primary hypertension), hypercholesterolemia/hyperlipidemia, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute ischemic attack, sudden cardiac death, peripheral vascular disease, coronary artery disease (CAD), carotid artery disease, peripheral artery disease (PAD), cerebrovascular disease, adverse ventricular remodeling, ventricular systolic dysfunction, ventricular diastolic dysfunction, cardiac dysfunction, ventricular arrhythmia, and stroke. In some embodiments, the disease or disorder comprises a disease or disorder associated with aging. In some embodiments, the disease or disorder is selected from non-alcoholic steatohepatitis (NASH), kidney disease, adverse ventricular remodeling, ventricular systolic dysfunction, ventricular diastolic dysfunction, cardiac dysfunction, and ventricular arrhythmia.

In some embodiments, if the method identifies that the subject has a risk of a cardiovascular disease or disorder, the method further comprises treating the subject with at least one treatment for cardiovascular disease. In some embodiments, the method comprises implementing a treatment regimen selected from an adjusted dietary regimen, an exercise regimen, administering a cholesterol lowering agent, administering a blood pressure modifying agent, or any combination thereof. In some embodiments, the method comprises administering to the subject an agent selected from a statin, a fibrate, niacin, a bile acid resin, a cholesterol absorption inhibitor, a phytosterol, an alginate, a pectin, lecithin, or nutraceutical. some embodiments, the method comprises administering to the subject an agent selected from Omega 3 oil, salicylic acid, dimethylbutanol, garlic oil, olive oil, krill oil, Co enzyme Q-10, a probiotic, a prebiotic, dietary fiber, psyllium husk, bismuth salts, phytosterols, grape seed oil, green tea extract, vitamin D, antioxidants, turmeric, curcumin, and resveratrol.

Also provided herein are compositions comprising, consisting of, or consisting essentially of sodium sulfide, wherein greater than 90% of the sodium sulfide is ³⁴S isotope-labeled sodium sulfide. In some embodiments, greater than 95% of the sodium sulfide is ³⁴S isotope-labeled sodium sulfide. In some embodiments, greater than 99% of the sodium sulfide is ³⁴S isotope-labeled sodium sulfide.

In some embodiments, the composition further comprises a basic buffer solution. In some embodiments, the basic buffer solution comprises ammonium bicarbonate. In some embodiments, the basic buffer solution has a pH of 9-11. In some embodiments, the basic buffer solution is ammonium bicarbonate at pH 10.

Additionally provided are methods of making ³⁴S isotope-labeled sodium sulfide. The methods comprise mixing elemental sulfur (³⁴S) and metallic sodium at about a 1:2 molar ratio; heating the mixture for greater than 18 hours; collecting resulting precipitate; and drying ³⁴S isotope-labeled sodium sulfide solid. In some embodiments, the ³⁴S isotope-labeled sodium sulfide solid has an isotopic abundance of greater than 90% ³⁴S. In some embodiments, the ³⁴S isotope-labeled sodium sulfide solid has an isotopic abundance of greater than 95% ³⁴S. In some embodiments, the ³⁴S isotope-labeled sodium sulfide solid has an isotopic abundance of greater than 99% ³⁴S.

Further provided herein kits comprising the composition as described herein and at least one or all of: a reducing agent, a derivatization agent, a chelating agent, an isotopically-labeled thiol-containing compound, a buffer, a solvent, and a container. In some embodiments, the reducing agent is an agent capable of reducing polysulfides. In some embodiments, the reducing agent is TCEP. In some embodiments, the derivatizing agent is selected from ethyl iodoacetate, ethyl bromoacetate, methyl bromoacetate, iodoacetate, and combinations thereof. In some embodiments, the derivatizing agent is ethyl iodoacetate. In some embodiments, the chelating agent is diethylenetriaminepentaacetic acid (DTPA). In some embodiments, the isotopically-labeled thiol-containing compound comprises [3,3-D₂] cysteine, [3,3,4,4-D₄] homocysteine, (¹³C₂¹⁵N) glutathione, or a combination thereof. In some embodiments, the kit comprises a sealable reaction vial. In some embodiments, the solvent is substantially free of sulfur-containing compounds.

Other aspects and embodiments of the disclosure will be apparent in light of the following detailed description.

### BRIEF DESCRIPTION OF THE DRAWINGS

FIGS. 1A-1D show the characterization and isotopic purity of commercially available Na₂S and synthesized isotope labelled internal standard [³⁴S]Na₂S. FIG. 1A is electrospray ionization (ESI, positive ion mode) high resolution mass spectrum of ethyl iodoacetate derivatized H₂S (top panel) and the corresponding derivative of synthesized internal standard ([³⁴S]H₂S) (bottom panel). FIG. 1B is the calculation of Δ (ppm) for H₂S derivatives (containing either ³²S or ³⁴S) observed in the high resolution mass spectra. FIG. 1C is the isotopic abundance of ³²S and ³⁴S in synthesized heavy isotope labelled internal standard ([³⁴S]Na₂S) and commercially available sodium sulfide (Na₂S).

FIG. 2 shows the chromatography and collision induced dissociation spectra of the derivatives of circulating H₂S and synthesized heavy isotope labelled [³⁴S]Na₂S. The chromatography (left panel) shows the retention time of 15.5 minutes for the derivatives of both H₂S and the heavy isotope labelled internal standard. The CID mass spectrum (right top panel) of the derivative of H₂S precursor ion (m/z=207.2) yields fragments (product ions) with m/z 77.0, 105.0, and 133.1, whereas the derivative of the heavy isotope labelled internal standard (right bottom panel, m/z=209.2) yields fragments with m/z 79.1, 107.1, and 135.1.

FIG. 3 shows circulating levels of total H₂S decrease with age. Older subjects show significantly lower levels of total H₂S in plasma than younger subjects. The age was stratified by four groups (>=45&<55,>=55&<65,>=65&<75,>=75). The division of subjects by gender shows that reduction of total H₂S with aging is independent of gender. Boxes indicate the 25^{th} and 75^{th} percentiles, respectively; the middle line is the median, and the lower and upper whiskers are the 5^{th} and 95^{th} percentiles, respectively. P-values were calculated using the Kruskal Wallis test where, p<0.05 is significant.

FIG. 4 shows the changes in levels of circulating total thiols with age. Older subjects show significantly lower levels of circulating total thiols compared to younger subjects in each of total cysteinylglycine, total glutamylcysteine and total glutathione. The downward trend is monotonic for total cysteinylglycine, total glutamylcysteine and total glutathione and visibly steeper for total cysteinylglycine. Boxes indicate the 25^{th} and 75^{th} percentiles, respectively, the middle line is the median, and the lower and upper whiskers are the 5^{th} and 95^{th} percentiles, respectively. P values were calculated using the Kruskal-Wallis test, where p<0.05 is significant.

FIGS. 5A-5C show the effect of gut microbiota suppression on circulating total H₂S levels. FIG. 5A is the circulating levels of total H₂S and thiols after administration of poorly absorbed broad-spectrum antibiotics. The circulating levels of total H₂S are reduced approximately 35% after antibiotics treatment. Conversely, the circulating levels of total cysteine, total homocysteine, total cysteinylglycine, total glutamylcysteine, and total glutathione do not change significantly following antibiotics administration. FIG. 5B is the circulating levels of total H₂S for individual subjects before antibiotics treatment displayed as white bars, and after antibiotics treatment as black bars. FIG. 5C is the percent reduction of the total H₂S circulating levels due to antibiotics treatment for individual subjects. The study shows that the reduction in total H₂S levels vary strongly among subjects. P values were calculated using the Mann-Whitney test where p<0.05 is significant.

FIGS. 6A-6D show optimization of the reaction conditions for sulfide derivatization with ethyl iodoacetate. FIG. 6A is a graph of the effect of various buffers on the yield of derivatization reaction; ammonium bicarbonate (NH₄HCO₃), ammonium formate (HCOONH₄), triethylamine ((C₂H₅)₃N). Ammonium bicarbonate buffer (pH >9.0) is preferred for derivatization. FIG. 6B is a graph of the yield of the derivatization reaction as a function of reaction time of sodium sulfide with ethyl iodoacetate. The yield reaches a plateau after 8 hours. FIG. 6C is a graph of the change in the yield of the derivatization reaction with the increase in the molar ratio of ethyl iodoacetate to Na₂S. The reaction appears to be nearly complete when the molar ratio of ethyl iodoacetate to H₂S reaches 1:80. FIG. 6D is a determination of the reductant concentration that fully reduces sulfide in samples used for H₂S quantification. The graph shows the change in the derivatization reaction (as estimated by the peak area of the sulfide derivative with ethyl iodoacetate) with the increase in the concentration of the reductant used (TCEP) when analyzing 20 μL of a human plasma pool spiked with 100 μM H₂S (to ensure that total H₂S level in the sample pool is at the upper end of what is observed within subject samples. The plateau observed in signal after approximately 5 mM TCEP indicates that the reaction is complete under the conditions described.

FIG. 7 shows collision induced dissociation spectra of derivatized thiols, and their heavy isotope labeled internal standard. CID mass spectra of ethyl iodoacetate derivatives of cysteine (m/z 208.3), homocysteine (m/z 221.5) and glutathione (m/z 394.1) in human plasma, and their corresponding heavy isotope labelled internal standards [3,3-D₂]-cysteine (m/z 210.3), [3,3,4,4-D₄]-homocysteine (m/z 225.6), [¹³C₂¹⁵N]-glutathione (m/z 397.1) respectively, and cysteinylglycine (m/z 265.4) and glutamylcysteine (m/z 337.1).

FIG. 8 shows the chromatography of heavy isotope labelled/nonlabelled thiols derivatized with ethyl iodoacetate. Chromatographic separation of thiol derivatives in human plasma and their corresponding isotope labeled counterparts used as internal standards. Top panel: reconstructed chromatograms from human plasma samples spiked with stable isotope labeled internal standards, cysteine, homocysteine, glutathione, and their corresponding isotope labeled counterparts ([3,3-D₂]-cysteine, [3,3,4,4-D₄]-homocysteine, (¹³C₂¹⁵N)-glutathione). Bottom panel: reconstructed chromatograms for glutamylcysteine and cysteinylglycine. Two MRMs of each is detected where the first MRM is used for quantification and the other is used for confirmation.

FIGS. 9A and 9B show selection of the polysulfide reducing reagent added to the buffer used for the derivatization reaction. FIG. 9A shows the amount of H₂S derivatized with ethyl iodoacetate (EIA) (displayed as peak area relative to EIA) measured without adding a polysulfide reducing reagent or after adding comparable molar excesses of either 10 mM dithiothreitol (DTT), 10 mM β-mercaptoethanol (BME) or 10 mM tris(2-carboxyethyl) phosphine (TCEP). A blank sample (containing only EIA in buffer) was derivatized as a negative control and compared to a plasma sample processed under same conditions (molar ratio H₂S: EIA 1:100, pH 10, at least 8 hours incubation at 21° C.). When TCEP was used as reducing reagent, the amount of derivatized H₂S detected in the blank sample (“Blank+TCEP”) was less than 5% of the amount detected in the plasma sample (“Plasma+TCEP”). On the other hand, the amount of H₂S detected in the blank sample with added DTT (“Blank+DTT”) was significantly elevated. Similarly, when TCEP versus a molar equivalent amount of either DTT or BME were added as reductant, the peak area for the derivatized H₂S measured was increased 1.67-fold, and 2.28-fold, respectively, indicating adventitious addition of H₂S from the commercial reductants. A blank sample without reducing reagent still produces a small amount of derivatized H₂S, which might be due to the water source or impurities present in the salts used to prepare the buffer solution. However, the levels observed are insignificant; <5% levels observed in plasma. FIG. 9B shows the capacity of TCEP to fully recover sulfide from polysulfide by first oxidizing the H₂S in the sample with 3% H₂O₂ and then by adding 10 mM TCEP to convert polysulfide back to sulfide, in gas tight sealed reaction vials with mininert caps. Following derivatization (H₂S: EIA molar ratio 1:80, pH 10, 21° C., at least 8 hours), H₂S was quantified by LC-MS/MS, and the result was compared with that obtained from an iodometric titration. The percentage of sulfide recovered by H₂S derivatization was normalized against the titration result. Briefly, aliquots of H₂S in buffer were oxidized by H₂O₂. An aliquot was treated with TCEP while the other was not; the TCEP treated sample showed 104% recovery whereas the non-treated sample produced only 33% recovery. Each derivatized sample was run in triplicates.

FIGS. 10A and 10B show interference of thiols with the quantification of H₂S in human plasma samples. Concentrations of derivatized H₂S (3 replicates) were measured in serum pool samples not spiked or spiked with thiols at their physiological or at 10 times their physiological concentrations, and a mix of thiols at their physiological concentrations or 10 times higher. FIG. 10A shows sulfide impurities were removed from the cysteine stock solution by acidifying with formic acid (2.5 mM in final sample volume), evaporation and reconstitution (e.g., H₂S was removed by off gassing during evaporation of the commercial thiol solutions). In FIG. 10B each thiol was spiked in the sample at its physiological concentration and at 10 times its physiological concentration, and then Total H₂S was quantified by LC-MS/MS to estimate the degree of interference with H₂S quantitation. The analysis of thiol spiked samples shows that the thiols do not interfere with the quantification of H₂S.

FIG. 11 are graphs showing the change in total thiol concentrations in male and female subjects with age. While older subjects showed significantly lower levels of total cysteinylglycine, total glutamylcysteine, and total glutathione than younger subjects, the same significance was not observed in neither total cysteine nor total homocysteine. The division of subjects by gender shows that reductions observed by age is gender dependent. MS analysis of total thiols in male samples showed a significant reduction with age, however a similar statistically significant reduction was not observed in samples from female subjects. Boxes indicate the 25^{th} and 75^{th} percentiles, respectively; the middle line is the median, and the upper and lower whiskers are the 5^{th} and 95^{th} percentiles, respectively. p values were calculated using the Kruskal Wallis test where p<0.05 is significant.

FIGS. 12A-12C are Kaplan Meier plot analyses of increasing tertile levels of total H₂S versus freedom from major adverse cardiac events (MACE=MI, stroke or death) event risk in the entire cohort (FIG. 12A) and in the indicated number of sequential subjects without congestive heart failure (FIG. 12B), and increasing tertile levels of total H₂S versus freedom from all-cause mortality in the entire cohort (FIG. 12C).

FIGS. 13A and 13B are graphs showing the hazard ration and the relationship to incident MACE over a 3 year period amongst subjects with and without congestive heart failure (CHF) or based on ejection fraction (EF) number (FIG. 13A) and with and without subclinical myocardial necrosis (elevated versus normal high sensitivity TnT level) or with (and without) evidence of subclinical myocardial strain (elevated versus normal NTproBNP level) (FIG. 13B). Adj: Age, gender, BPSystolic, DIABETICS and Current Smoker.

### DETAILED DESCRIPTION

Provided herein are methods and compositions that can detect and quantify total H₂S and multiple thiols in biological matrices by stable isotope dilution mass spectrometry (e.g., LC-MS/MS). [³⁴S]Na₂S was synthesized as internal standard and used to develop a stable isotope dilution mass spectrometry method (e.g., isotope-labeled liquid chromatography tandem mass spectrometry), coupled with the use of a reducing agent such as tris(2-carboxyethyl) phosphine hydrochloride (TCEP) to reduce and derivatize free and reversibly oxidizable sulfur-containing compounds, for the simultaneous quantification of total H₂S and thiols in biological matrices. Use of a reducing agent that is substantially free of H₂S, such as TCEP, allows for recovery of protein-bound and mixed disulfide and persulfide forms of sulfide, without artefactual addition of sulfide. Beyond normal range studies, plasma H₂S and six abundant thiols were examined in a clinical cohort (n=400), and in subjects before and after suppression of gut microbiota.

Using the disclosed methods and compositions, all analytes showed minimal interference, no carryover, and excellent intra- and inter-day reproducibility (≤7.6%, and ≤12.7%, respectively), linearity (r²>0.997), recovery (90.9%-110%) and stability (90.0%-100.5%). Only circulating total H₂S levels showed significant age-associated reductions in both males and females (p<0.001), and a marked reduction following gut microbiota suppression (mean 33.8±17.7%, p<0.001), with large variations in gut microbiota contribution among subjects (range 6.0 to 66.7% reduction with antibiotics). Overall, total H₂S levels were significantly reduced with aging, and gut microbiota contribution to circulating total H₂S was shown to vary significantly between subjects.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of cell and tissue culture, molecular biology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, “chelating agent” includes any compound which complexes with metal ions. In the present context the term “chelating agents” is used interchangeably with “complexing agent,” “chelator,” “chelant,” or “sequestering agent.” Examples of suitable chelating agents include citric acid, nitrilotriacetic acid (NTA), any form of ethylene diamine tetraacetic acid (EDTA), diethylene triamine pentaacetic acid (DTPA), propylene diamine tetraacetic acid (PDTA), ethylene diamine-N,N″-di(hydroxyphenyl) acetic acid (EDDHA), ethylene diamine-N,N″-di-(hydroxy-methylphenyl) acetic acid (EDDHMA), ethanol diglycine (EDG), trans-1,2-cyclohexylene dinitrilotetraacetic acid (CDTA), glucoheptonic acid, gluconic acid, sodium citrate, phosphonic acid, and salts thereof. In some embodiments, the chelating agent may be a sodium or potassium salt.

As used herein, “derivatizing” means reacting two molecules to form a new molecule. Thus, a “derivatizing agent” is an agent that is reacted with another substance to derivatize the substance. Generally, a specific functional group of the compound participates in the derivatization reaction and transforms the compound to a derivate with improved physicochemical properties, which can be used for the quantification or separation of the original compound. Derivatization typically involves silylation, alkylation, or acylation. The derivatizing agents selected for use preferably generate derivatized analyte compounds that are distinguishable by mass spectrometry. Derivatizing agents may include isothiocyanate groups, dansyl groups, dinitro-fluorophenyl groups, nitrophenoxycarbonyl groups, phthalaldehyde groups, and alpha-halocarbonyl groups.

The term “electrospray ionization,” or “ESI,” as used herein refers to methods in which a solution is passed along a short length of capillary tube, to the end of which is applied a high positive or negative electric potential. Upon reaching the end of the tube, the solution may be vaporized (nebulized) into a jet or spray of very small droplets of solution in solvent vapor. This mist of droplet can flow through an evaporation chamber which is heated slightly to prevent condensation and to evaporate solvent. As the droplets get smaller the electrical surface charge density increases until such time that the natural repulsion between like charges causes ions as well as neutral molecules to be released.

As used herein, “isotope dilution mass spectrometry” refers to a mass spectrometry analytical technique based on the modification of the natural isotope composition of analytes in a sample following the addition of an enriched isotope or an isotopically labeled form of the analyte(s). The ratio of the quantity of unlabeled to labeled compound is measured, and the concentration of the original analyte in the sample can be determined.

As used herein, “liquid chromatography” or “LC” means a process of selective retardation of one or more components of a fluid solution as the fluid uniformly percolates through a column of a finely divided substance, or through capillary passageways. The retardation results from the distribution of the components of the mixture between one or more stationary phases and the bulk fluid, (e.g., mobile phase), as this fluid moves relative to the stationary phase(s). Liquid chromatography includes reverse phase liquid chromatography (RPLC), high performance liquid chromatography (HPLC) and high turbulence liquid chromatography (HTLC).

The term “reducing agent,” as used herein, refers to and includes a substance that causes another substance to undergo reduction and that is oxidized in the process. A reducing agent may serve to keep compounds in a reduced state and to prevent oxidation thereof. Examples of reducing agents include dithiothreitol (DTT), tris(2-carboxyethyl) phosphine hydrochloride (TCEP), lithium aluminum hydride (LiAlH₄), sodium borohydride (NaBH₄), diborane, beta mercaptoethanol (BME), and diisobutylaluminum hydride (DIBAL-H).

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the term “substantially free” means that recited compounds or compositions contain less than an measurable or detectable amount of the recited contaminant. For example, “substantially free of H₂S” indicates that the compound or composition (e.g., an isotope-labeled thiol compound) contains less than a detectable amount of H₂S as determined using the same analytical assay as for the target sample.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Methods for Detecting Hydrogen Sulfide

Disclosed herein are methods and systems for the analysis hydrogen sulfide, and optionally, thiol containing compounds. In some embodiments, the methods and systems may be used for the quantitative analysis of total levels of hydrogen sulfide in a sample. Total hydrogen sulfide represents a summation of sulfide forms that are able to be recovered following reduction, for example, free hydrogen sulfide plus the sum of reversibly oxidized forms like persulfated proteins and mixed disulfides. In some embodiments, the methods and system may be used for the quantitative analysis of total levels of thiol containing compounds, including, for example, cysteine, homocysteine, glutathione, cysteinylglycine and glutamylcysteine.

In some embodiments, the methods comprise adding an internal standard to the sample (e.g., prior to derivatization). For example, in some embodiments, the internal standard is an isotopically-labeled derivative of the analyte of interest (e.g., sulfide or thiol compound). In some embodiments, the methods comprise adding a ³⁴S isotope-labeled compound, such as ³⁴S isotope-labeled sodium sulfide, to the sample. Preferably, the ³⁴S isotope-labeled sulfide compound has a higher isotopic abundance of ³⁴S to differentiate it from the endogenous forms of sulfide from the sample. In some embodiments, the ³⁴S isotope-labeled sulfide compound (e.g., ³⁴S isotope-labeled sodium sulfide) has an isotopic abundance of greater than 90% ³⁴S (e.g., greater than 91%, greater than 92%, greater than 93%, greater than 94%, greater than 95%, greater than 96%, greater than 97%, greater than 98%, greater than 99%, greater than 99.5% ³⁴S). In some embodiments, the ³⁴S isotope-labeled sulfide compound, such as ³⁴S isotope-labeled sodium sulfide, is produced by the method described herein.

In some embodiments, the methods comprise adding an isotope labeled thiol compound to the sample (e.g., prior to derivatization). The isotope labeled thiol compound may comprise one or more or all of: [3,3-D₂] cysteine, [3,3,4,4-D₄] homocysteine, and [¹³C₂¹⁵N] glutathione. For example, when the method is being used to detect cysteine, [3,3-D₂] cysteine is the isotope labeled thiol compound used as the internal standard; when the method is being used to detect homocysteine or cysteinylglycine, [3,3,4,4-D₄] homocysteine is the isotope labeled thiol compound used as the internal standard; and when the method is being used to detect glutathione or glutamylcysteine, [¹³C₂¹⁵N] glutathione is the isotope labeled thiol compound used as the internal standard.

In some embodiments, the isotope labeled thiol compounds were pretreated to remove residual sulfide impurities. Removal of the residual sulfide impurities may be completed by acidifying a solution containing the thiol compounds and evaporating under vacuum, to remove hydrogen sulfide gas, until the solid thiol compounds were formed. The solid thiol compounds can be reconstituted in a desired buffer or solvent.

The internal standard may be added to the sample in a basic buffer solution. In some embodiments, the basic buffer solution may comprise ammonium bicarbonate, ammonium formate, or triethylamine. In select embodiments, the basic buffer solution comprises ammonium bicarbonate. In some embodiments, the basic buffer solution has a pH greater than 7, greater than 8, greater than 9, greater than 10, or greater than 11. In select embodiments, the pH is between 9 and 11. In exemplary embodiments, the pH is 10. In select embodiments, the basic buffer solution is ammonium bicarbonate at pH 10.

In some embodiments, the methods comprise adding a reducing agent to the sample (e.g., prior to derivatization). In some embodiments, the internal standard(s) (e.g., ³⁴S isotope-labeled sodium sulfide or isotopically labeled thiol compound) and the reducing agent are added simultaneously, e.g., at the same time from a single source or multiple sources to the sample. In some embodiments, the internal standard(s) and the reducing agent are added separately, e.g., separated by a period of time, to the sample.

The method is not limited by the type or types of reducing agent employed. In preferred embodiments, the reducing agent is capable of reducing polysulfides. In select embodiments, the reducing agent is tris(2-carboxyethyl) phosphine (TCEP).

In some embodiments, the reducing agent is added to the sample for a final concentration of at least 1 mM (e.g., at least 2 mM, at least 3 mM, at least 5 mM, at least 10 mM, at least 15 mM, at least 20 mM, or more). In some embodiments, the final concentration of the reducing agent is 1-100 mM, or 1-20 mM. The final concentration of the reducing agent may be 1-15 mM, 1-10 mM, 1-5 mM, 1-2 mM, 2-20 mM, 2-15 mM, 2-10 mM, 2-5 mM, 10-20 mM, 10-15 mM, or 15-20 mM. In exemplary embodiments, the final concentration of the reducing agent is 12.5 mM.

In some embodiments, the methods further comprise adding a chelating to the sample. The chelating agent may be added before, after, or concomitantly with the reducing agent and/or the internal standard. In some embodiments, the chelating agent is diethylenetriaminepentaacetic acid (DTPA). Generally, the chelating agent may be present in an amount sufficient to prevent undesired side effects of divalent or trivalent cations that may be present in the sample.

In some embodiments, the methods comprise incubating the sample with a derivatizing agent to form a derivatized sample.

The derivatizing agent is not particularly limited as long as it reacts with the analytes to be measured and increases ionization efficiency or enhances detection sensitivity. In some embodiments, the derivatization agent has high reactivity with sulfhydryl or thiol groups. The derivatizing agent may be selected from ethyl iodoacetate, ethyl bromoacetate, methyl bromoacetate, and iodoacetate. In some embodiments, the derivatizing agent is ethyl iodoacetate.

In some embodiments, the derivatizing agent is present in a molar excess compared to hydrogen sulfide in the sample. In some embodiments, the derivatizing agent is at a molar ratio of hydrogen sulfide to derivatizing agent of at least about 1:10. In some embodiments, the hydrogen sulfide to derivatizing agent molar ratio ranges from 1:10 to 1:100. The molar ratio of hydrogen sulfide to derivatizing agent may be about 1:10, about 1:20, about 1:30, about 1:40, about 1:50, about 1:70, or about 1:100.

In some embodiments, the derivatizing agent is at a final concentration of 1-100 mM. The final concentration of the derivatizing agent may be about 1 mM, about 10 mM, about 20 mM, about 30 mM, about 40 mM, about 50 mM, about 60 mM, about 70 mM, about 80 mM, about 90 mM or about 100 mM. In some embodiments, the derivatizing agent is at a final concentration of about 10 mM.

The incubation time for derivatization may be any period of time that facilitates complete or nearly complete derivatization of hydrogen sulfide or sodium sulfide, such that the products can be detected by mass spectrometry. The incubation time may be from 2 to 24 hours (e.g., 2-8 hours, 2-12 hours, 2-16 hours, 2-20 hours, 8-12 hours, 8-16 hours, 8-20 hours, 8-24 hours, 12-16 hours, 12-20 hours, 12-24 hours, 16-20 hours, 16-24 hours, or 20-24 hours. In some embodiments, the incubation time for derivatization is at least about 8 hours.

The incubation temperature for derivatization may be any temperature that facilitates complete or nearly complete derivatization of hydrogen sulfide or sodium sulfide, such that the products can be detected by mass spectrometry. In some embodiments, the temperature ranges from 10-60° C. The temperature may be about 10° C., about 20° C., about 30° C., about 40° C., about 50° C., about 60° C. In some embodiments, the incubation temperature is room temperature (20-23° C.)

Given the internal standards are provided to the sample prior to derivatization, the internals standards may also undergo derivatization along with the endogenous analytes. In this case, ions of the derivatized internal standards are detected by mass spectrometry.

In some embodiments, the methods further comprise reducing the proteins in the derivatized sample. For example, reducing the proteins in the derivatized sample may comprise precipitating proteins in the derivatized sample and removing the precipitated proteins from the derivatized sample. Any method of precipitating proteins may be used with the disclosed methods. For example, the proteins can be precipitated by salting out or addition of a neutral salt, changing the pH of the solution (isoelectric precipitation), addition of miscible solvents (e.g., alcohols, acetonitrile), addition of polymers (e.g., dextrans and polyethylene glycol), or addition of polyelectrolytes (e.g., alginate, carboxymethylcellulose, polyacrylates).

### A. Liquid Chromatography & Tandem Mass Spectrometry

In certain embodiments, the methods and systems of the present invention comprise analyzing the derivatized sample by liquid chromatography in combination with mass spectrometry. For example, in one embodiment, the present invention comprises a method for determining the presence or amount of an analyte (e.g., derivatized sulfide or thiol compound) in a sample comprising chromatographically separating the analyte from other components in the sample using reverse phase liquid chromatography. The method may also comprise analyzing the chromatographically separated analytes by mass spectrometry to determine the presence or amount in the sample.

Liquid chromatography (LC) separate analytes of interest from one another in a mixture of compounds, or from other constituents in a test sample, for the purpose of identifying, quantifying, and/or purifying the individual components of the mixture. Liquid chromatography typically comprises the use of a high performance liquid chromatography (HPLC) column. However, any column that can sufficiently resolve the analytes of interest (e.g., unlabeled sulfides or thiol compounds) and allow for detection according to the method can be employed.

Mass spectrometry (MS) generally refers to methods of filtering, detecting, and measuring ions based on their mass-to-charge ratio (m/z). In mass spectrometry, one or more molecules of interest are ionized, and the ions are subsequently introduced into a mass spectrometer where, due to a combination of magnetic and electric fields, the ions follow a path in space that is dependent upon their mass (m) and charge (z). In some embodiments, a tandem mass spectrometry (MS/MS) system is used. Tandem mass spectrometry is a group of mass spectrometric methods wherein parent or precursor ions generated from a sample are fragmented to yield one or more fragment or product ions, which are subsequently mass analyzed by a second MS procedure. The analyte of interest may then be quantified based upon the amount of the characteristic transitions measured by tandem MS.

Examples of suitable mass separators include, but are not limited to, quadrupoles, RF multipoles, ion traps, time-of-flight (TOF), and TOF in conjunction with a timed ion selector. In some embodiments, the tandem mass spectrometer comprises a triple quadrupole mass spectrometer. Methods of ionization such as the use of inductively coupled plasma, electrospray ionization (ESI), matrix-assisted laser desorption ionization (MALDI), atmospheric pressure chemical ionization (APCI), or atmospheric pressure photoionization (APPI) may be used for ionization. In some embodiments, the methods use a triple quadrupole tandem mass spectrometer with an electrospray ionization (ESI) source operating in positive ion mode.

In some embodiments, the methods comprise multiple reaction monitoring (MRM).

Multiple reaction monitoring (MRM) involves selecting a precursor ion for fragmentation and monitoring the fragmentation for a specific fragment ion, or product ion. For example, when using a triple quadrupole mass spectrometer, the first quadrupole, herein referred to as Q1, acts as a first mass separator. The transmitted mass-to-charge (m/z) range of Q1 is selected to transmit a molecular ion, often referred to as the “parent ion” or the “precursor ion,” to the second quadrupole, herein referred to as Q2. This can be accomplished, for example, by setting Q1 to transmit ions in a mass window about 3 mass units wide substantially centered on the mass of a proteolytic fragment. Q2 acts as an ion fragmentor (e.g., a collision cell, photodissociation region, etc.) that can be maintained at a sufficiently high pressure and voltage so that multiple low energy collisions occur, producing fragment ions, often referred to as “daughter ions.” Q2 can comprise a collision gas for conducting collision-induced dissociation (CID) and a quadrupole to facilitate the collection and transmittal of daughter ion fragments to a third quadrupole, referred to herein as Q3. Q3 acts as a second mass separator. The transmitted m/z range of Q3 is selected to transmit one or more daughter ions to a detector which measures the daughter ion signal. This can be accomplished by setting Q3 to transmit ions in a mass window about 1 mass unit wide substantially centered on the mass of a daughter ion.

Using MRM analysis, multiple analytes can be monitored in single run. A monitored pair of parent ion and daughter ion masses can be referred to as an MRM transition. Where a parent ion is generated and the ion signal of the corresponding daughter ion is measured, the daughter ion signal at the detector is referred to as the “MRM transition signal.” The MRM transition signal can be based on the intensity (e.g., the average, mean, maximum, etc.) of the daughter ion peak, the integrated area of the daughter ion peak, or any combination thereof.

MRM transitions are identified by two numbers that correspond to a first and a second m/z value, respectively, separated by “→,” “>,” or “/” (e.g., 207.2→133.1, 207.2>133.1 or 207.2/133.1). That is, the first value corresponds to the precursor ion, and the second value corresponds to the product ion after fragmentation of the precursor ion in the collision cell. Depending on the sensitivity of the mass spectrometer, some variability is possible for the transitions provided in this disclosure (e.g., ±0.1-0.5).

The MRM transitions may be used to quantitate the selected analyte (e.g., unlabeled derivatized sulfide or unlabeled thiol compounds) by comparing the peak areas to that of a labeled internal standard (e.g., ³⁴S isotope-labeled derivatized sulfide or isotope labelled thiol compounds, respectively). The area ratio is the ratio of the peak area of the unlabeled analyte to the peak area of the labeled internal standard. With the known concentration of the labeled internal standard, the analyte can be quantified by calculated by multiplying the concentration with the area ratio. The area ratio, and by extension, the concentration of the analyte can be confirmed by calculating the area ratio of the secondary MRM transitions.

In some embodiments, the primary MRM for unlabeled derivatized sulfide is 207.2→133.1. In some embodiments, the secondary MRM for unlabeled derivatized sulfide is 207.2→105.0.

In some embodiments, the primary MRM for ³⁴S isotope-labeled derivatized sulfide is 209.2→135.1. In some embodiments, the secondary MRM for ³⁴S isotope-labeled derivatized sulfide is 209.2→107.1.

In some embodiments, the methods further comprise detecting additional thiol species in the sample. In some embodiments, the methods further comprise detecting primary and secondary MRM transitions for one or more or all of: cysteine, homocysteine, glutathione, cysteinylglycine and glutamylcysteine; calculating an area ratio of the primary MRM transition for one or more or all of: cysteine, homocysteine, glutathione, cysteinylglycine and glutamylcysteine to a corresponding isotope labeled thiol compound primary MRM transition; and determining a concentration by multiplying the concentration of corresponding isotope labeled thiol compound with the area ratio.

In some embodiments, the primary MRM for unlabeled cysteine is 208.3→163.1. In some embodiments, the secondary MRM for unlabeled cysteine is 208.3→88.9.

In some embodiments, the primary MRM for unlabeled homocysteine is 221.5→102.2. In some embodiments, the secondary MRM for unlabeled homocysteine is 221.5→134.1.

In some embodiments, the primary MRM for unlabeled glutathione is 394.1→174.2. In some embodiments, the secondary MRM for unlabeled derivatized glutathione is 394.1→219.1.

In some embodiments, the primary MRM for unlabeled cysteinylglycine is 265.4→115.9. In some embodiments, the secondary MRM for unlabeled cysteinylglycine is 265.4→162.1.

In some embodiments, the primary MRM for unlabeled glutamylcysteine is 337.1→191.1. In some embodiments, the secondary MRM for unlabeled glutamylcysteine is 337.1→309.1.

In some embodiments, the primary MRM for [3,3-D₂] cysteine is 210.3→165.1. In some embodiments, the secondary MRM for [3,3-D₂] cysteine is 210.3→91.0.

In some embodiments, the primary MRM for [3,3,4,4-D₄] homocysteine is 225.6→106.1. In some embodiments, the secondary MRM for [3,3,4,4-D₄] homocysteine is 225.6→138.1.

In some embodiments, the primary MRM for (¹³C₂¹⁵N) glutathione is 397.4→177.1. In some embodiments, the secondary MRM for (¹³C₂¹⁵N) glutathione is 397.4→222.1.

b. Sample

As used herein, the term “sample” is used in its broadest sense. In one sense, it is meant to include a specimen obtained from any source, including biological and environmental samples, and in vitro laboratory or analytical samples. Biological samples may be obtained from animals (including humans) and encompass fluids, solids, and/or tissues. Such examples are not however to be construed as limiting the sample types. In some embodiments, the sample is a fluid sample such as a liquid sample. Examples of liquid samples suitable for use with the devices disclosed herein include bodily fluids (e.g., blood, serum, plasma, saliva, urine, ocular fluid, semen, sputum, sweat, tears, and spinal fluid), water samples (e.g., samples of water from oceans, seas, lakes, rivers, and the like), samples from home, municipal, or industrial water sources, runoff water, or sewage samples; and food samples (e.g., milk, beer, juice, or wine). Viscous liquid, semisolid, or solid specimens may be used to create liquid solutions, eluates, suspensions, or extracts that can be samples. Liquid samples can be made from solid, semisolid, or highly viscous materials, such as fecal matter, tissues, organs, biological fluids, or other samples that are not fluid in nature. For example, solid or semisolid samples can be mixed with an appropriate solution, such as a buffer, a diluent, and/or extraction buffer. The sample can be macerated, frozen and thawed, or otherwise extracted to form a fluid sample. Residual particulates may be removed or reduced using conventional methods, such as filtration or centrifugation. Samples can comprise biological materials, such as cells, microbes, organelles, and biochemical complexes. In some embodiments, the samples are cell-free, microbe-free, and/or organelle-free.

The biological sample may be obtained from any suitable subject, typically a mammal (e.g., dogs, cats, rabbits, mice, rats, goats, sheep, cows, pigs, horses, non-human primates, or humans). Preferably, the subject is a human. The sample may be obtained from any suitable biological source, such as, a physiological fluid including, but not limited to, whole blood, serum, plasma, interstitial fluid, saliva, ocular lens fluid, cerebral spinal fluid, sweat, urine, milk, ascites fluid, mucous, synovial fluid, peritoneal fluid, vaginal fluid, menses, amniotic fluid, semen, feces, and the like. In some embodiments, the sample is blood or blood products. Blood products are any therapeutic substance prepared from human blood. This includes whole blood; blood components (e.g., red blood cell concentrates or suspensions; platelets produced from whole blood or via apheresis; plasma; serum and cryoprecipitate); and plasma derivatives (e.g., coagulation factor concentrates).

In some embodiments, the sample is a biological sample obtained from a subject having or suspected of having a disease or disorder. In some embodiments, samples are obtained from a subject throughout the course of a disease or disorder or during treatment for a disease or disorder and the samples are analyzed for changes in the hydrogen sulfide, and/or thiol compounds, over the time period of sample collection.

The sample can be obtained from the subject using routine techniques known to those skilled in the art, and the sample may be used directly as obtained from the biological source or following a pretreatment to modify the character of the sample. Such pretreatment may include, for example, preparing plasma from blood, diluting viscous fluids, filtration, precipitation, dilution, distillation, mixing, concentration, inactivation of interfering components, the addition of reagents, lysing, and the like.

c. Predicting Disease Risk

The methods may further comprise predicting risk of a disease or disorder or death in a subject, such as cardiovascular disease or a disease associated with aging. Predicting the risk may comprise comparing the hydrogen sulfide concentration, as determined by the disclosed method, to a control hydrogen sulfide concentration. In some embodiments, an increased hydrogen sulfide concentration indicates an increased risk of the disease or disorder.

In some embodiments, the disease or disorder are associated with aging-aging-associated conditions. By aging-associated condition is meant a condition, e.g., a disease condition or other undesirable condition, which accompanies aging of an organism. The aging associated condition may manifest in a number of different ways, e.g., as aging-associated cognitive impairment and/or physiological impairment, e.g., as aging associated damage to central or peripheral organs of the body, such as but not limited to: cell injury, tissue damage, organ dysfunction, aging-associated lifespan shortening and carcinogenesis, where specific organs and tissues of interest include, but are not limited to skin, neuron, muscle, pancreas, brain, kidney, lung, stomach, intestine, spleen, heart, adipose tissue, testes, ovary, uterus, liver and bone. In some embodiments, the disease or disorder associated with aging is selected from impaired cognitive function, Alzheimer's Disease, arthritis (e.g., osteoarthritis or rheumatoid arthritis), osteoporosis, macular degeneration, dementia, and Type 2 diabetes.

In some embodiments, the disease or disorder comprises cardiovascular disease. For example, the cardiovascular disease may be angina, arrhythmia, arteriosclerosis, atherosclerosis, myocardial infarction, acute coronary syndrome, cardiomyopathy, congestive heart failure, coronary thrombosis, aortic aneurysm, aortic dissection, iliac or femoral aneurysm, pulmonary embolism, high blood pressure/hypertension (e.g., primary hypertension), hypercholesterolemia/hyperlipidemia, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute ischemic attack, sudden cardiac death, peripheral vascular disease, coronary artery disease (CAD), carotid artery disease, peripheral artery disease (PAD), cerebrovascular disease, adverse ventricular remodeling, ventricular systolic dysfunction, ventricular diastolic dysfunction, cardiac dysfunction, ventricular arrhythmia, and stroke. In select embodiments, the disease or disorder comprises myocardial infarction or stroke.

d. Methods of Treatment

The methods may further one or more treatment methods for a disorder, such as a cardiovascular disease or a disease or disorder associated with aging. For example, if the methods described above identify that the subject has a risk of a cardiovascular disease or disorder, then in some embodiments, the method further comprises treating the subject with at least one treatment for cardiovascular disease.

Examples of therapies that may be used for treating a subject with increased risk of cardiovascular disease include an adjusted dietary regimen, an exercise regimen, administering a cholesterol lowering agent, administering a blood pressure modifying agent, or any combination thereof. Examples of particular cardiovascular disease therapies include but are not limited to, statins (e.g., Lipitor™ (atorvastatin), Pravachol™ (pravastatin), Zocor™ (simvastatin), Mevacor™ (lovastatin), and Lescol™ (fluvastatin)) or other agents that interfere with the activity of HMGCoA reductase, nicotinic acid (niacin, which lowers LDL cholesterol levels), fibrates (which lower blood triglyceride levels and include, for example Bezafibrate (such as Bezalip®), Ciprofibrate (such as Modalim®), Clofibrate, Gemfibrozil (such as Lopid®) and Fenofibrate (such as TriCor®)), bile acid resins (such as Cholestyramine, Colestipol (Colestid), and Cholsevelam (Welchol)), cholesterol absorption inhibitors (such as Ezetimibe (Zetia®, Ezetrol®, Ezemibe®)), phytosterols such as sitosterol (Take Control (Lipton)), sitostanol (Benechol), or stigmastanol), alginates and pectins, lecithin, and nutraceuticals (such as extract of green tea and other extracts that include polyphenols, particularly epigallocatechin gallate (EGCG), Cholest-Arrest™ (500 mg garlic and 200 mg lecithin). Cholestaway™ (700 mg Calcium carbonate, 170 mg magnesium oxidem 50 μg chromium picolinate), Cholest-Off™ (900 mg of plant sterols/stanols), Guggul Bolic (750 mg gugulipid (Commiphora mukul gum resin), and Kyolic® (600 mg aged garlic extract and 380 mg lecithin)). In some embodiments, the methods further comprise administering an agent selected from Omega 3 oil, salicylic acid, dimethylbutanol, garlic oil, olive oil, krill oil, Co enzyme Q-10, a probiotic, a prebiotic, dietary fiber, psyllium husk, bismuth salts, phytosterols, grape seed oil, green tea extract, vitamin D, antioxidants, turmeric, curcumin, and resveratrol.

### ³⁴S Isotope-Labeled Sodium Sulfide

Further disclosed herein are compositions comprising, consisting of, or consisting essentially of sodium sulfide, wherein greater than 90% (e.g., greater than 91%, greater than 92%, greater than 93%, greater than 94%, greater than 95%, greater than 96%, greater than 97%, greater than 98%, greater than 99%, greater than 99.5%) of the sodium sulfide is ³⁴S isotope-labeled sodium sulfide. In some embodiments, the compositions comprise, consist of, or consist essentially of greater than 95% ³⁴S isotope-labeled sodium sulfide. In selected embodiments, the compositions comprise, consist of, or consist essentially of greater than 99% ³⁴S isotope-labeled sodium sulfide.

In some embodiments, the compositions further comprise a buffer. In some embodiments, the buffer may comprise ammonium bicarbonate, ammonium formate, or triethylamine. In select embodiments, the buffer comprises ammonium bicarbonate. In some embodiments, the buffer has a pH greater than 7. Thus, the buffer may be a basic buffer solution. In some embodiments, the pH is greater than 8, greater than 9, greater than 10, or greater than 11. In select embodiments, the pH is between 9 and 11. In exemplary embodiments, the pH is 10. In some embodiments, the compositions further comprise an ammonium bicarbonate buffer at pH 10.

Also provided herein are methods of making ³⁴S isotope-labeled sodium sulfide. The methods may comprise mixing elemental sulfur (³⁴S) and metallic sodium at about a 1:2 molar ratio; heating the mixture at 80° C. for greater than 18 hours; collecting resulting precipitate; and drying ³⁴S isotope-labeled sodium sulfide solid.

In some embodiments, the reaction comprises a catalyst (e.g., naphthalene). The reaction may be completed under an anhydrous conditions (e.g., nitrogen atmosphere).

The precipitate may be collected by filtration, centrifugation, decantation, and the like. The resulting precipitate may be washed in a suitable solvent (e.g., water, alcohol, etc.). The precipitate may be dried by any method including, but not limited to, oven drying, air drying, vacuum drying, desiccation, and freeze drying.

In some embodiments, the ³⁴S isotope-labeled sodium sulfide solid has an isotopic abundance of greater than 90% (e.g., greater than 91%, greater than 92%, greater than 93%, greater than 94%, greater than 95%, greater than 96%, greater than 97%, greater than 98%, greater than 99%, greater than 99.5%).

### Kits

Also within the scope of the present disclosure are systems and kits that include ³⁴S isotope-labeled sodium sulfide and at least one or all of: a reducing agent, a derivatization agent, a chelating agent, an isotopically-labeled thiol-containing compound, a buffer, a solvent, and a container.

In some embodiments, the ³⁴S isotope-labeled sodium sulfide has an isotopic abundance of greater than 90% ³⁴S (e.g., greater than 91%, greater than 92%, greater than 93%, greater than 94%, greater than 95%, greater than 96%, greater than 97%, greater than 98%, greater than 99%, greater than 99.5% ³⁴S). In some embodiments, the ³⁴S isotope-labeled sodium sulfide is produced by the method described herein.

The kit is not limited by the type(s) of reducing agent. In preferred embodiments, the reducing agent is capable of reducing polysulfides. In select embodiments, the reducing agent is tris(2-carboxyethyl) phosphine (TCEP).

The kit is not limited by the type(s) of derivatization agent. In some embodiments, the derivatization agent has high reactivity with sulfhydryl or thiol groups. The derivatizing agent may be selected from ethyl iodoacetate, ethyl bromoacetate, methyl bromoacetate, and iodoacetate. In some embodiments, the derivatizing agent is ethyl iodoacetate.

The kit is not limited by the type(s) of chelating agent. In some embodiments, the chelating agent is diethylenetriaminepentaacetic acid (DTPA).

The kit is not limited by the type(s) of containers. In some embodiments, the kit comprises a sealable reaction vial.

In some embodiments, the kit comprises a buffer or solvent that is substantially free of sulfur-containing compounds.

The isotope labeled thiol compound may comprise one or more or all of: [3,3-D₂] cysteine, [3,3,4,4-D₄] homocysteine, and (¹³C₂¹⁵N) glutathione.

The kit may include instructions for use in any of the methods described herein. Instructions supplied in the kits of the disclosure are typically written instructions on a label or package insert. The materials may include any combination of the following: background information, list of components and their availability information (purchase information, etc.), brief or detailed protocols for using the compositions, troubleshooting, references, technical support, and any other related documents. Instructions can be supplied with the kits or as a separate member component, either as a paper form or an electronic form which may be supplied on computer readable memory device or downloaded from an internet website, or as recorded presentation.

Individual member components of the kits may be physically packaged together or separately. The components of the kits may be provided in bulk packages (e.g., multi-use packages) or single-use packages. The kits provided herein are in suitable packaging. Suitable packaging includes, but is not limited to, vials, bottles, jars, flexible packaging, and the like.

Kits optionally may provide additional components. Normally, the kit comprises a container and a label or package insert(s) on or associated with the container. In some embodiment, the disclosure provides articles of manufacture comprising contents of the kits described above. In some embodiments, the systems kits may further comprise the materials or hardware for LC-MS/MS including LC columns, matrix components, mobile phase components, and the like. In some embodiments, the kits may further contain materials for procuring or processing the sample.

It is understood that the disclosed systems or kits can be employed in connection with the disclosed methods.

### EXAMPLES

The following are examples of the present invention and are not to be construed as limiting.

### Materials and Methods

Materials Screwcap cryovials (1.5 mL) were purchased from Sigma-Aldrich. Aeris 2.6 μm Peptide XB-C18 100 LC column 150×4.6 mm, Security guard Ultra Holder, Security guard Ultra Cartridges were purchased from Phenomenex (Torrance, CA). Chemglass Life Sciences 25 mL Tube, Storage, 14/20 Outer Joint, Airfree, Schlenk tube and 2 mL Filter Funnel, Buchner, Fine Frit were purchased from Thermo Fisher Scientific (Waltham, MA). All reagents and mobile phases were LC-MS-grade solvents purchased from Thermo Fisher Scientific (Waltham, MA) unless otherwise specified. MS grade water was produced in-house by a Millipore Milli-Q purification system with an LC-Pak Polisher filter for ultrapure water used for HPLC and LC-MS (Darmstadt, Germany).

Reagents and chemicals All reagents and mobile phases were LC-MS-grade solvents (methanol, acetonitrile, formic acid) were purchased from Thermo Fisher Scientific (Waltham, MA) unless otherwise specified. MS grade water was produced in-house by a Millipore Milli-Q purification system with an LC-Pak Polisher filter for ultrapure water used for HPLC and LC-MS (Darmstadt, Germany). Cysteine (99.2%), glutathione (TLC ≥98%), and TCEP (0.5 M) were from Thermo Fisher Scientific (Waltham, MA). Homocysteine (>98.0%) was from Tokyo Chemical Industry. Sodium sulfide (97.0-103.0% by titration), γ-glutamylcysteine (≥80% (HPLC)), cysteinylglycine (>85% (TLC)), DTT (≥97% (Ellman's reagent)), and BME (≥99.0%) were from Sigma-Aldrich (St. Louis, MO). Commercially available tetrahydrofuran (≥99.9%), naphthalene, formic acid (>99%), ethyl iodoacetate (98%), methyl bromoacetate (97%), ethyl bromoacetate (98%), iodoacetic acid (≥98.0%) and DTPA (>99.0%) were from Sigma-Aldrich (St. Louis, MO). Isotope-labelled internal standards DL-homocysteine (3,3,4,4-D₄, 98%), L-cysteine (3,3-D₂, 98%), glutathione (95%+) (Glycine-¹³C₂, 98%+;¹⁵N, 96-99%) 90%+ net peptide were from Cambridge Isotopes (Andover, MA). Ammonium bicarbonate (99%) and ammonium formate (99%) were from Sigma-Aldrich (St. Louis, MO), and trimethylamine (99.5%) was from Thermo Scientific Pierce.

Research subjects All subjects gave written informed consent, all study protocols were approved by the Institutional Review Board of the Cleveland Clinic and adhered to the Declaration of Helsinki principles. Subject antibiotics exposure followed an approved protocol registered at ClinicalTrials.gov (NCT01731236). Samples for establishing a normal range of analytes were collected from non-fasting subjects undergoing community health screening. From these, a random subset of subjects (n=200) was selected who met the inclusion criterion requirements of no medical history of cardiometabolic diseases or cancer, normal vital signs, and normal metabolic test values on screening studies (lipid profile, complete metabolic panel).

Blood was drawn from subjects for all studies, including those comparing the effect of vacutainer tube type on analyte levels, using a 21 Gauge BD Vacutainer Safety-Lok Blood Collection Set. Plasma vacutainer tubes were placed on ice or within a refrigerator until processing, and serum tubes were maintained at room temperature for 60 minutes to allow for clotting to proceed before centrifugation. One hour after blood collection, both plasma and serum vacutainer tubes were spun for 15 minutes at 4° C. at 1530 relative centrifugal force. Samples were aliquoted into 1.5 mL O-ring screw cap cryovials (catalogue number Z353361, Millipore Sigma, St. Louis, MO), placed immediately on dry ice, and then stored at −80° C. until analysis.

Synthesis and storage of heavy isotope labelled internal standard [³⁴S]Na₂S Heavy isotope-labelled sodium sulfide ([³⁴S]Na₂S) was synthesized using the reaction of metallic sodium and elemental sulfur ³⁴S in the presence of naphthalene as a catalyst (So J H, et al., Inorganic Syntheses 1992:30-2). The reaction was performed under anaerobic conditions under dry nitrogen atmosphere in an anhydrous and degassed medium within a glovebox (MBraun Labstar Pro). All glassware were dried at 160° C. prior to use. Tetrahydrofuran was deoxygenated by sparging with dry nitrogen, and then dried via passage through activated alumina in an MBraun MB-SPS solvent purification system. Briefly, elemental sulfur (³⁴S) and metallic sodium were mixed in a 1:2 molar ratio inside the glove box in a Schlenk tube at room temperature. The Schlenk tube was sealed, and then heated in an oil bath (outside the glovebox) until color change to white (80° C., 24 hours). The Schlenk tube was then moved back into the glovebox, and the precipitate was collected on filter paper, and dried overnight under nitrogen atmosphere. The purity of the heavy isotope label sulfide ([³⁴S]Na₂S) was confirmed by iodometric titration and the isotopic enrichment for [³⁴S] (versus other isotopologues) was quantified by high resolution mass spectrometry.

The internal standard stock solution was prepared by weighing anhydrous [³⁴S]Na₂S (in glovebox under nitrogen atmosphere) and dissolving it in ammonium bicarbonate buffer (pH 10). Once dissolved in aqueous solution at basic pH, the highly charged sulfide ion internal standard has low vapor pressure and is more stable (losses from outgassing are minimal). The stock solution was stored in conventional o-ring sealed cryovials (500 μL with minimal headspace gas) at −80° C., and the concentration of this stock was confirmed by either iodometric titration (as described), or by the method of standard addition using authentic anhydrous [³²S]Na₂S.

Sample preparation During methods development and throughout the derivatization reaction optimization process, samples were handled in gas tight vials with mininert caps. While the reaction was carried out, the IS mix and derivatizing reagent were injected into the tube through the septa using gas-tight Hamilton syringes.

Serum samples were blanketed with argon and stored at −80° C. After thawing at 4° C., samples were aliquoted (20 μL) on dry ice into screw cap O-ring tubes. The IS mixture master stock (500 μL in a 500 μL cryovial) was stored at −80° C. with minimal headspace gas. The IS mixture used in sample preparation was prepared from IS mixture master stock, and before opening vials were thawed on ice. The reducing reagent (TCEP) was added to the sample at the same time as the internal standard mix (prior to derivatization). Endogenous H₂S (free and oxidized, including protein bound) and thiols, as well as their respective internal standards, were reduced by treating samples with TCEP prior to derivatization.

To derivatize samples (20 μL), ethyl iodoacetate solution (50 μL, 16 mM) and IS mixture (10 μL) were added (final concentrations in final volume (80 μL) are 6.25 mM ammonium bicarbonate (pH 10.0), 12.5 mM TCEP, 10 mM EIA), vials were sealed and vortexed for 2 minutes, and then left at room temperature (21° C.) for least 8 hours (and no longer than 24 hours) to allow the derivatization reaction to complete. Before analysis, proteins were precipitated with ice cold acetonitrile (100 μL), samples centrifuged (14,000×g, 20 min, −1° C.), and supernatants transferred to HPLC vials.

LC-MS/MS run conditions Analysis of the derivatized samples was performed on an AB SCIEX LC-MS/MS 4000 Q-Trap triple quadrupole tandem mass spectrometer with an electrospray ionization (ESI) source in positive ion mode. Separation of analytes was performed on a reverse phase column (Aeris™ 2.6 μm Peptide XB-C18 100 Å, LC Column 150×4.6 mm, Phenomenex, Torrance, CA). Mobile phase A (5 mM ammonium formate, 0.2% formic acid in water), and mobile phase B (0.2% formic acid in 25:75 v/v methanol:acetonitrile) were used for discontinuous gradient separations.

The concentration of each analyte was determined by the area ratio obtained from a primary MRM transition (e.g., for H₂S 207.2→133.1 ([³²S] isotopologue) to 209.2→135.1 ([³⁴S] isotopologue)) and then quantification was confirmed by obtaining comparable results using the area ratio of secondary MRM transitions (207.2→105.0 (for [³²S] isotopologue) and 209.2→107.1 (for [³⁴S] isotopologue)), and a similar approach was applied for other thiols with their respective heavy isotope labelled internal standards. Commercially available internal standards for the dipeptide analytes cysteinylglycine and glutamylcysteine could not be obtained, therefore [3,3,4,4,-D₄]-homocysteine and [¹³C₂,¹⁵N]-glutathione were used as internal standards for cysteinylglycine and glutamylcysteine, respectively (MRM transitions 225.6→106.1 for [3,3,4,4,-D₄]-homocysteine, 265.4→162.1 for cysteinylglycine, 397.4→177.1 for [¹³C₂,¹⁵N]-glutathione, 337.1→191.1 for glutamylcysteine).

Establishing reference range of analytes Analyte normal ranges were determined based on standards defined from CLIA and the College of American Pathologists. The reference range was determined to be the 95% central interval bounded between the 2.5^{th} and 97.5^{th} percentiles and used to determine the lower and upper limit concentrations in the listed concentrations.

Selecting the reagent for the derivatization of H₂S Derivatizing reagents that were commercially available were sought after in order to make the assay of total H₂S quantification robust and applicable to large cohorts of samples. Accordingly, several chemical compounds with iodide or bromide as a leaving group were tested in the reaction, including ethyl iodoacetate (EIA), ethyl bromoacetate (EBA), methyl bromoacetate (MBA), and iodoacetate (IA). The derivatization reactions for tested reagents are shown below:

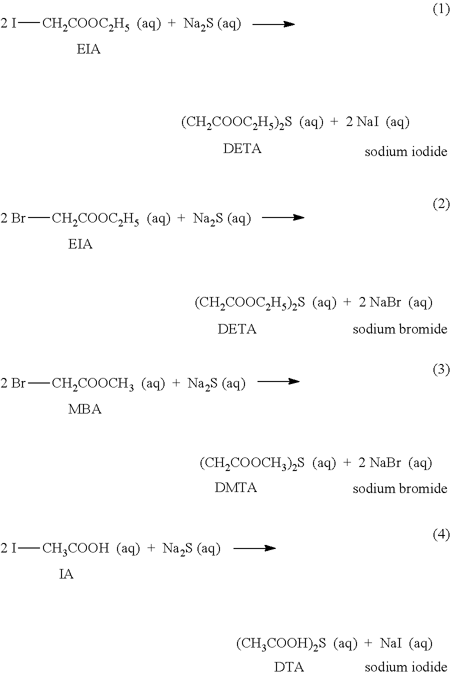

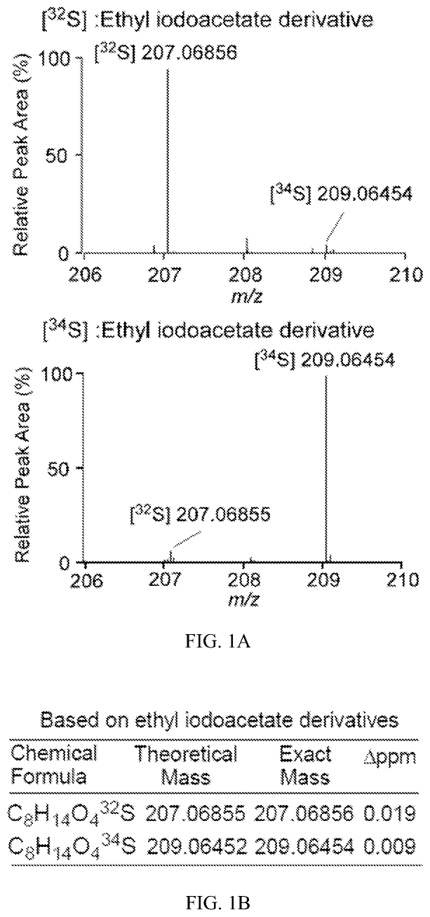

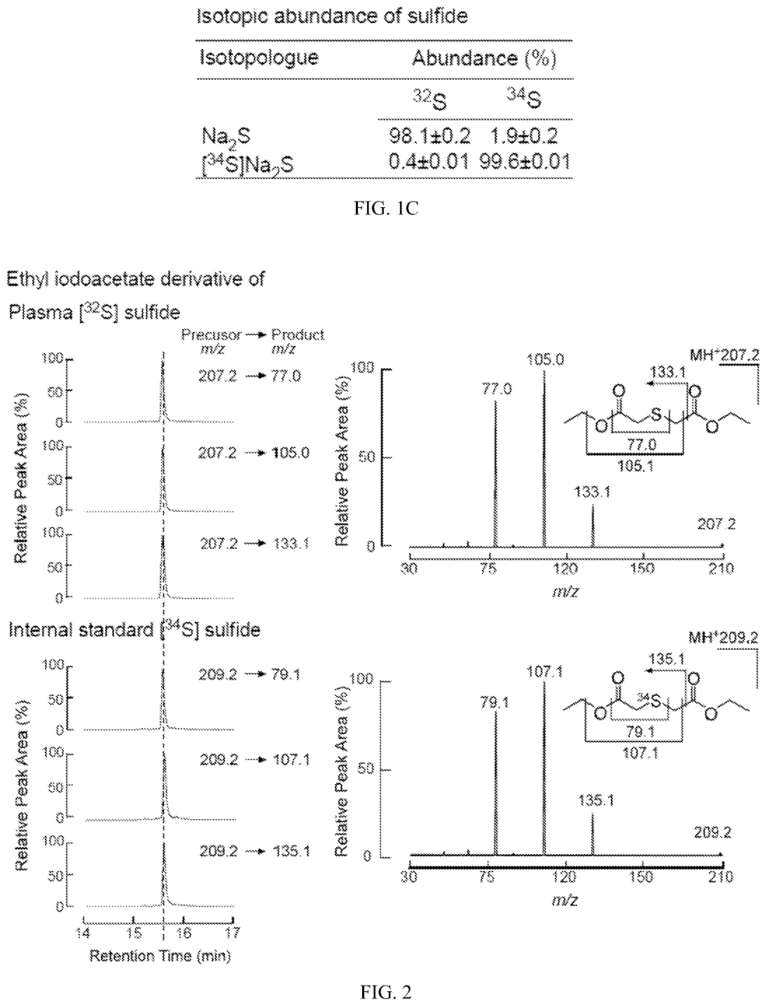

The reaction of Na₂S with each of these reagents was carried out at 21° C. and pH 8.0 for 24 hours, at a molar ratio of Na₂S to derivatizing reagent of 1:10. The H₂S derivatized with each reagent was separated and detected by LC-MS/MS, and the yield of each derivatization reaction (relative to EIA) was estimated from the peak area of the parent ion. Table 2 lists the peak areas of the MS1 ions of H₂S derivatized with all reagents tested and concludes that EIA is the most effective in derivatizing the sulfide ion, which was therefore selected as the derivatizing reagent for the development of the stable isotope dilution LC-MS/MS method described here. At the molar ratio of H₂S to EIA used (1:10) each sulfide ion was expected to react with 2 molecules of EIA producing 2,2′-diethyl thiodiacetate (DETA) with a theoretical molecular weight of 206.1 a.u. Fragmentation of the precursor ion (m/z=207.2, FIG. 1) in positive ion mode results in 3 product ions whose precursor→product ion transitions are 207.2→77.0, 207.2→105.0, and 207.2→133.1 (FIG. 2, top-left panel). The product ion with m/z 133.1 was used for quantification while product ions with m/z 77.0 and 105.0 were used for confirmation, respectively. The isotope-labelled internal standard (e.g., the derivative of [³⁴S]H₂S synthesized in this work, vide infra) was similarly derivatized and fragmented (FIG. 1, bottom-right panel), resulting in the following precursor→product ion transitions: 209.2→79.1, 209.2→107.1, and 209.2→135.1 (FIG. 1, bottom-left panel, fragmentation of the precursor ion is shown in FIG. 1, bottom-right panel).

Optimization of the reaction conditions for the derivatization of H₂S with ethyl iodoacetate and the determination of the yield for the derivatization reaction The overall optimized reaction conditions used for the final assay method employed when analyzing a 20 μL aliquot of sample (serum or plasma) in a total reaction volume of 80 μL was as follows (final concentrations): 6.25 mM ammonium bicarbonate (pH 10.0), 12.5 mM TCEP, 10 mM EIA at 21° C., for a derivatization time of at least 8 hours (though up to 24 hours data shows comparable results for H₂S and all thiols monitored in the panel). Isotope labeled internal standards were also included (prior to reductant and derivative addition) at the final concentrations: 10 μM [³⁴S]Na₂S, 10 μM DL-homocysteine (3,3,4,4-D₄, 98%), 50 μM L-cysteine (3,3-D₂, 98%), 10 μM glutathione (95%+) (Glycine-¹³C₂, 98%+;¹⁵N, 96-99%) 90%).

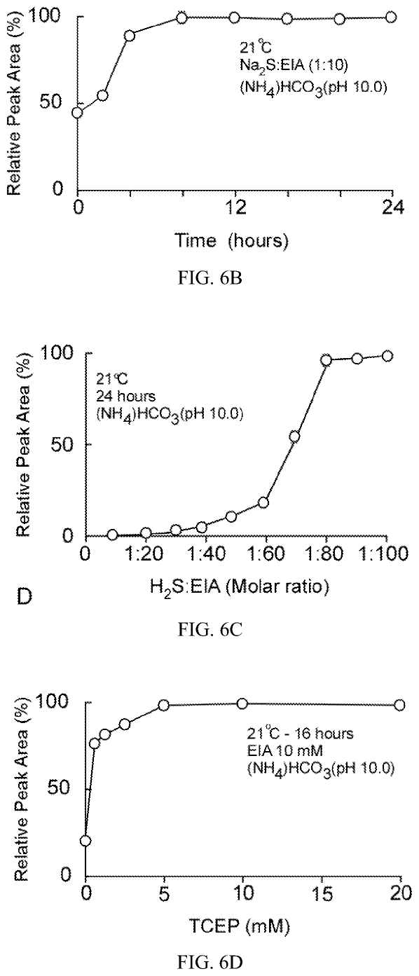

LC-MS/MS was used to estimate the amount of derivatized H₂S produced in the reaction while varying a number of parameters (e.g., buffer, pH, temperature, reaction time, and the H₂S: EIA molar ratio). Since H₂S is a gas at room temperature and normal pressure, it is only partially soluble in water, and in general is partitioned between gas phase and solution. The equilibria in water between neutral (H₂S) and ionic (HS⁻, S²⁻) forms of H₂S depend on both temperature and pH. Published data on H₂S solubility indicated that the amount of H₂S present in solution increased with pH, as increasingly alkaline conditions cause neutral H₂S to convert to ionic forms. Accordingly, the effect of several buffers was tested, including ammonium bicarbonate (NH₄HCO₃), ammonium formate (HCOONH₄), and triethylamine ((CH₃CH₂)₃N), to determine the yield of H₂S derivatization within the pH range of 8.0-10.0. The derivatized H₂S produced through the reaction with EIA (H₂S: EIA molar ratio 1:10, at 21° C. for 24 hours) as a function of pH is shown in FIG. 6A as relative peak area of the product ion (MS2, m/z 133.1) of 2,2′-diethyl thiodiacetate (DETA). The ammonium bicarbonate buffer (pH 10) was found to produce the highest amount of derivatized H₂S in the pH range 8-10, and accordingly was selected for optimizing the other reaction conditions (temperature, reaction time, and the H₂S: EIA molar ratio). At room temperature and pH 8, 10% of H₂S is in gas form while at pH 10 less than 1% of H₂S is gaseous.

To evaluate the optimal reaction temperature, the derivatization reaction with EIA was carried out at 21° C., 37° C., and 60° C. (H₂S: EIA molar ratio 1:10 at pH 10 for 24 hours). The increase in temperature shortened the reaction time, but did not produce a significant change in the yield of H₂S derivatization; moreover, while a slightly larger peak area was observed for the derivative of H₂S, the higher temperature resulted in split peaks for the derivatized cysteine. Room temperature was used for derivatization in order to optimally quantify both H₂S and circulating thiols in the same run. To evaluate the time required for the derivatization reaction to reach maximum yield, Na₂S (100 μM) was reacted with EIA (H₂S: EIA molar ratio 1:100) at 21° C. in ammonium bicarbonate buffer (pH 10) for durations from 5 minutes to 24 hours. After 8 hours the yield of H₂S derivatization reaches a plateau (FIG. 6B)—suggesting that the reaction reached completion at 8 hours and the yield did not change between 8 and 24 hours. Finally, the derivatization was performed at ten different H₂S: EIA molar ratios ranging from 1:10 to 1:100. As shown in FIG. 6C, the amount of derivatized H₂S begins to plateau at a H₂S: EIA molar ratio of approximately 1:80, which was selected and used in all subsequent studies.

To evaluate the amount of reductant (TCEP, vide infra) required to fully reduce oxidized H₂S, the sample aliquots were sequentially spiked with 0-20 mM TCEP and the reaction with 10 mM EIA at 21° C. in ammonium bicarbonate buffer (pH 10) proceeded for 16 hours. As seen in FIG. 6D the plateau in peak area begins at 5 mM TCEP. 12.5 mM TCEP was used in the final assay to reduce both the endogenous H₂S and thiols and their internal standards (e.g., internal standard mix) in the samples. To summarize, the following conditions were established for the derivatization reaction: room temperature (21° C.), pH 10, overnight incubation (at least 8 hours and no longer than 24 h), the molar ratio of H₂S to EIA of 1:80, and reductant concentration (TCEP) of 5 mM.

The yield for the derivatization reaction for H₂S and thiols was determined using the standard addition method wherein samples were analyzed without addition, versus increased amounts of unlabeled H₂S (1.5, 3, 6, 12, 24, 48, 96 μM). The quantified concentration of total H₂S present in the sample was calculated by linear regression of the best fit line, with a (negative) X intercept indicating the endogenous amount present (e.g., in Table 1-9.4 μM for QC1). The same approach was done to determine the yield of the derivatization reaction for all thiols in the panel. The spiked concentrations of the analytes (H₂S or other thiols) in the standard addition method spanned within the range of endogenous values observed across subject samples for total H₂S and for total levels of each thiol.

In addition, a colorimetric assay (iodometric titration) based on iodine reduction to iodide by titration with thiosulfate was used to determine the purity of sulfide ([³²S]Na₂S and [³⁴S]Na₂S used in establishing the assay) by precipitating the sulfide ion with zinc acetate (2N). Then, iodine solution (0.025 N) was gradually added to each sample of derivatized sulfide until the color changed to amber yellow, and then to dark blue by adding starch. Next, the samples were titrated with thiosulfate solution (0.025 N) until colorless. The titration was done in 3 replicates. The following chemical reactions occur in the iodometric titration:

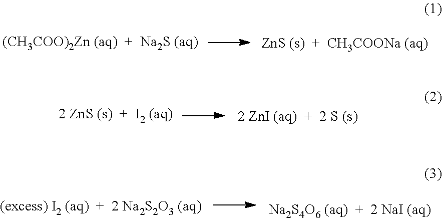

The sulfide (mg) was determined from the volumes and concentrations of iodine and thiosulfate solutions used in titration according to the following formula (3): Sulfide (mg)=[(Volume I₂ (mL)×Normality I₂)−(Volume Na₂S₂O₃ (mL)×Normality Na₂S₂O₃)×(32.06/2)]; Sulfide purity=100×(1-Measured residual sulfide/Sulfide in analyzed sample).

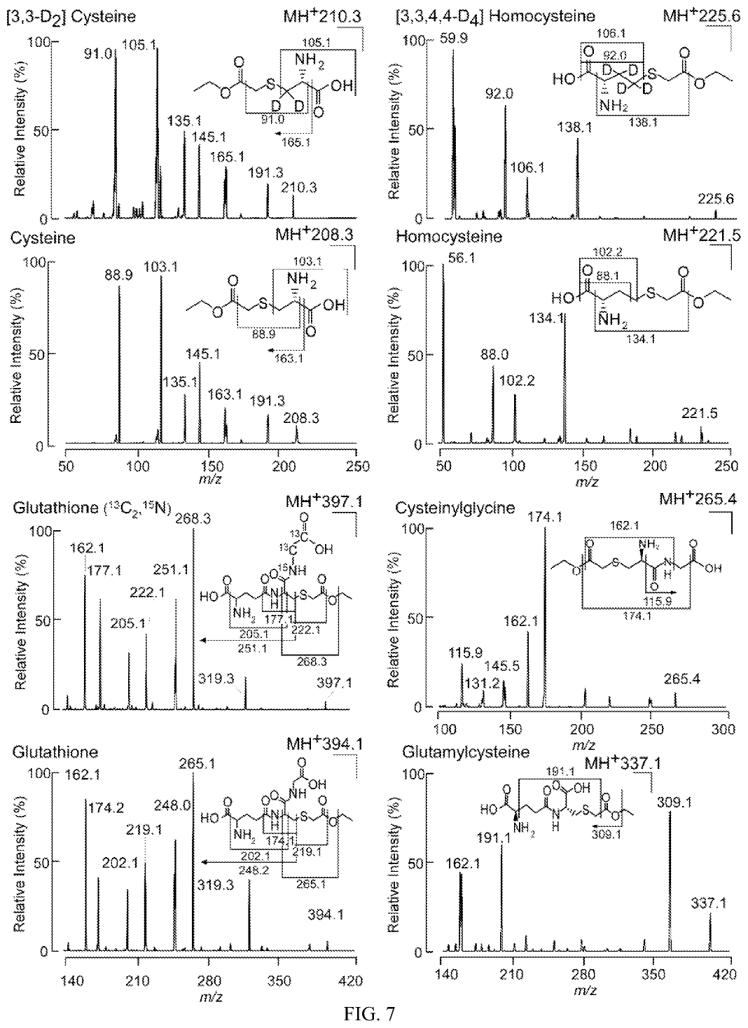

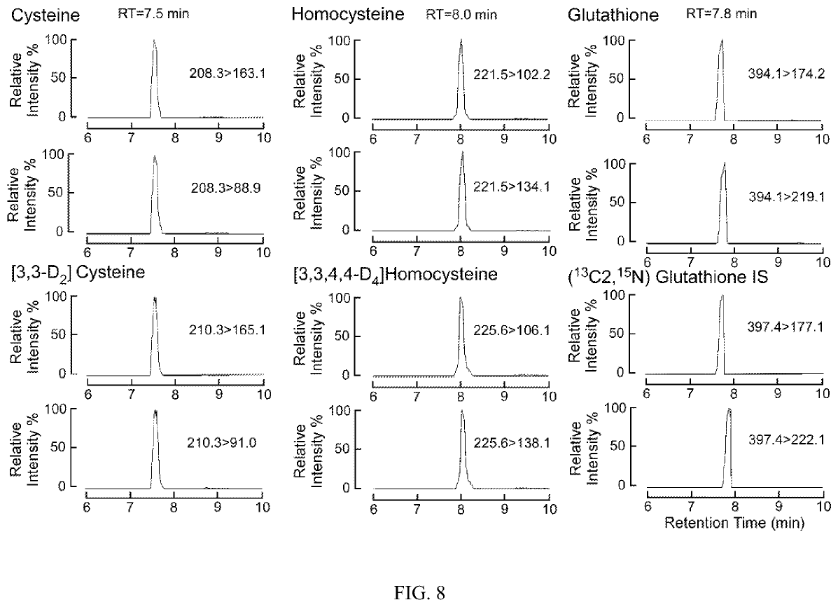

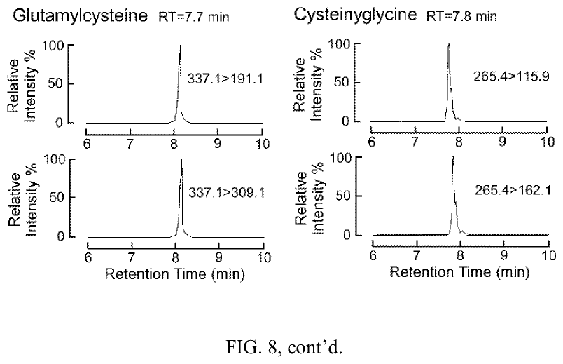

The same reaction conditions were used to derivatize thiols found in plasma. The thiols (cysteine, homocysteine, glutathione, cysteinylglycine and glutamylcysteine) were derivatized with EIA (pH 10, 21° C., overnight incubation) in parallel to their respective isotope labelled internal standard ([3,3-D₂] cysteine, [3,3,4,4-D₄] homocysteine, (¹³C₂¹⁵N) glutathione). The CID mass spectra (FIG. 7) show the fragmentation of each thiol derivative. Using the same separation conditions (reverse phase chromatography), thiols were sequentially retained on the MS column and monitored using characteristic parent→daughter ion transitions as indicated below and in FIG. 8.

Synthesis and characterization of the heavy isotope labelled internal standard [³⁴S]Na₂S Since commercially available heavy isotope elemental ³⁴S might not be isotopically pure (>99.0% indicated by the manufacturer), the isotopic purity of the synthesized internal standard was determined to ensure correct quantitation of H₂S in plasma samples. Samples of isotope labelled internal standard ([³⁴S]Na₂S) and commercially available Na₂S were derivatized with EIA (using optimal reaction conditions) and analyzed using a LC-Q Exactive LC-MS/MS (Hybrid Quadrupole-Orbitrap) (FIG. 2). For each sample the peak areas of two ions, with m/z of 207.06856 and 209.06452 corresponding to the derivatives of H₂S and [³⁴S]H₂S, respectively, were compared (FIG. 1A). Differences (Δ, ppm) between the measured mass and theoretical mass are 0.019 for the derivative of H₂S and 0.009 ppm for the derivative of [³⁴S]H₂S (FIG. 1B). The isotopic abundance determined for commercially available natural abundance Na₂S was 98.1±0.2% [³²S] and 1.9±0.2% [³⁴S], while the isotopic abundance for our IS (synthesized [³⁴S]Na₂S) was 0.4-0.01% [³²S] and 99.6-0.01% [³⁴S] (FIG. 1C).

Preparation of standards and internal standards used for LC-MS/MS quantitation Prior to the preparation of the stock working standards, the compounds used for stock solutions were tested for presence of residual sulfide (which can interfere with H₂S quantitation). Each thiol used as standard was quantified by LC-MS/MS spanning its physiological concentrations, and at 10 times its physiological concentration, to determine whether its presence interferes with H₂S quantitation. During these studies it was found, for example, that commercially acquired cysteine (regardless of source, and also when purchased as stable isotope standard) had a high content of residual sulfide (sufficient to significantly interfere with the assay). Sulfide impurities were first removed from all thiol stocks (standards, and natural abundance solutions used for spike and recovery studies) by acidifying with formic acid (2.5 mM in final sample volume), evaporation under vacuum until dry, and then reconstitution (e.g., H₂S gas was removed by evaporation). The purity of the stock thiol solutions was ultimately validated by comparing gravimetrically prepared acidified versus non-acidified stock solutions of commercial standards. After removing the sulfide impurities, thiol standards were added to a stock master solution as described above.

Next, stock solutions of working standards (including heavy isotope labeled internal standards (IS) of thiols (DL-homocysteine (3,3,4,4-D₄, 98%), L-cysteine (3,3-D₂, 98%), glutathione (95%+)), were prepared in ammonium bicarbonate buffer (pH 10). An IS stock master solution was prepared and aliquoted into screwcap O-ring cryovials with minimal headspace, blanketed with argon and stored at −80° C. until use (IS composition: 100 μM [³⁴S]Na₂S, 100 μM DL-homocysteine (3,3,4,4-D₄, 98%), 500 μM L-cysteine (3,3-D₂, 98%), 100 μM glutathione (95%+) (Glycine-¹³C₂, 98%+;¹⁵N, 96-99%) 90%). A stock master solution was prepared and aliquoted into screwcap O-ring cryovials with minimal headspace, blanketed with argon, and stored at −80° C. until use (standard composition: 96 μM Na₂S, 180 μM DL-homocysteine, 1600 μM cysteine, 80 μM glutathione, 120 μM cysteinylglycine, 24 μM γ-glutamylcysteine). IS working solutions were diluted 10-fold immediately before use. The IS and standard master solutions stored at −80° C. for 12 months showed no significant degradation. The concentration of the internal standard aqueous stock can be confirmed by either iodometric titration, or the method of standard addition using authentic dry [³⁴S]Na₂S.

Selection of polysulfide reducing reagent and prevention of contaminant sulfide introduction in preparing samples for H₂S quantification Both endogenous oxidants and post-collection oxidation processes can convert sulfide into polysulfide and mixed disulfides, artificially reducing the amount of derivatized H₂S and thereby skewing quantification. To avoid this, all samples were reduced to liberate and measure total H₂S. To avoid artificial oxidation of H₂S during sample preparation by redox active transition metal ions, or alternatively, its non-enzymatic generation catalyzed by iron, diethylenetriaminepentaacetic acid (DTPA) was added to solutions used for sample preparation in molar excess to divalent cation levels (Mg²⁺, Ca²⁺, and trace levels of active metal ions present in plasma and serum). In early studies, when using reducing reagents like β-mercaptoethanol (BME) and dithiothreitol (DTT) at levels needed (empirically determined) to quantitatively reduce polysulfides in plasma samples, significant artificial elevation in total H₂S was observed due to trace contaminant H₂S in the commercially acquired reductants. The frequent current use of alternative thiol based reductants including DTT and BME led to potential contamination because of sulfide presence in commercially available reductants. This was thereafter avoided and polysulfides were shown to be quantitatively recovered as free reduced H₂S, using tris(2-carboxyethyl) phosphine (TCEP) as the reductant. Similarly, during spike and recovery studies with thiols abundant in the circulation and monitored within the panel, significant artificial elevation of H₂S levels was seen when using commercial sources of thiols at physiological concentrations. After developing protocols to remove sulfide impurities in commercial thiols, the assay methods developed showed quantitative recovery of each analyte within the panel without interference or artificial elevation of H₂S levels, even when spiked with thiols at levels 10 times higher than those observed (in human plasma) under physiological conditions.

Stability of samples containing H₂S and thiols For stability analysis a fresh serum sample was divided into 5 equal aliquots and frozen at −80° C. The aliquots were thawed and analyzed 24 hours later. This process was repeated five times. Percent recovery (stability) was calculated with the following formula: % Stability=100×Cₜ/C₀; where C₀ is the initial concentration (μM) of total H₂S/thiols in the fresh serum sample and Cₜ is the concentration after each freeze-thaw cycle. The concentration mean from the 5 freeze-thaw cycles was calculated along with the standard deviation for each compound.

Long-term stability of H₂S and thiols in plasma samples was determined by “accelerated” stability. Samples were incubated in gas tight vials at 60° C. Each day, for five days (and after 1 year), an aliquot was cooled on ice, derivatized, and then analyzed by mass spectrometry. Stability was reported as the mean of the daily stability assay calculated across the five days. The long-term stability was calculated using the following formula: % Stability=(Cₜ/C₀)×100; where C₀ is the initial concentration of thiols and Cₜ is the concentration after each day of incubation. To determine whether the analyte was stable following derivatization, samples from 3 QC levels were kept at auto-sampler temperature and tested for five days. Stability was reported as the mean of the daily stability results for five days using the following formula: % Stability=(Cₜ/C₀)×100; where C₀ is the initial concentration of thiols and Cₜ is the concentration after each day of incubation.

Conditions for running samples on LC-MS/MS Analysis of the derivatized samples was performed by running the samples on an AB SCIEX LC-MS/MS 4000 Q-Trap triple quadrupole tandem mass spectrometer with an electrospray ionization (ESI) source operating in positive ion mode. The mass spectrometer running parameters were as follows: ion spray voltage 4200 V, ion source heater temperature 500° C., source gas-1 35 psi, source gas-2 45 psi, and curtain gas 35 psi. Nitrogen was used as the nebulizer, curtain, and collision gas. The optimal collision energies and declustering potentials of each thiol were set based on the response from the direct infusion of each standard solution in methanol. Each thiol was monitored using ESI in positive-ion mode with multiple reaction monitoring (MRM) of the precursor and its corresponding fragment ion. The HPLC system consisted of two binary pumps (LC-20 AB), an auto-sampler (Nexera X2 SIL-30AC) set to operate at 10° C., and a CBM-20A controller (Shimadzu Scientific Instruments, Inc.). Separation of analytes via chromatography was performed on a reverse phase column (Aeris™ 2.6 μm Peptide XB-C18 100 Å, LC Column 150×4.6 mm, Phenomenex, Torrance, CA). Mobile phase A was composed of 5 mM ammonium formate and 0.2% formic acid in water, and mobile phase B was 0.2% formic acid in 25:75 v/v methanol:acetonitrile mixture. The method takes 22 minutes. In order to keep the mass spectrometer clean the run was diverted to waste at the beginning (5.5 minutes) and at the end (5 minutes) of each run, scanning for the analytes monitored for 11.5 minutes (between 5.5-17 minutes in the run). The sample (10 μL) was injected into the column equilibrated in 100% A, and analytes were separated with the following gradient: 0% B for 0-1 min; 0-50% B for 1-2 min; 50% B for 2-8 min, 50-100% B for 8-13 min, and 100% B for 13-17 min; 0% B 17.1-22 min. The flow rate was 0.3 mL/min, and analytes eluted into the mass spectrometer from 5.5 to 17 min.

Accuracy, precision, recovery, LLOD, LLOQ, and matrix effect Blood samples were collected from subjects, centrifuged, aliquoted and stored at −80° C. in rapid time frame (typically within one hour of collection). Sample stability at different stages of sample collection, preparation, and analytical process, as well as, internal standards storage, and generation of sulfide-free thiols used in spike and recovery studies, alternative internal standards, etc. were investigated in order to increase the robustness of the method.

Calibration curves were generated using the spike and recovery approach, prepared using water and run with the samples. Calibration curve concentrations had at least 2 points above and below the reference range, and were built from the values determined from peak areas of analyte/IS. QCs were used to determine accuracy and precision. Concentrations of total H₂S and thiols were determined using the standard addition method (SAM) in each QC level. The accuracy was determined by comparing the SAM concentration to the mean concentration of each QC sample (QC mean concentration), calculated from six replicates. Accuracy is reported as percentage: % Accuracy=[(SAM concentration−QC mean concentration)/SAM concentration]×100. Precision was calculated as imprecision by comparing six different replicates of each QC using the following formula: Imprecision (coefficient of variation, %)=100×(Standard Deviation)/(QC mean concentration) (Table 1). Six calibration curves prepared in each matrix (water, methanol and acetonitrile) were tested against six different human serum pools spiked with increased concentrations of H₂S and the 6 thiols. Percentage matrix effect (% ME) was calculated using the following formula: % ME=(average slope of serum pool calibration curve/average slope of matrix matched calibration curve)×100. Water was a better matrix match to human serum pools than methanol and acetonitrile.

The lower limit of detection (LLOD) was defined as the lowest concentration of analyte in a sample matrix (e.g., serum) that generated a signal-to-noise ratio equal to or greater than 3. The lower limit of quantitation (LLOQ) was defined as the lowest concentration of analyte in a sample matrix that generated a signal-to-noise ratio of equal to or greater than 10.

Determining the contribution of gut microbiota to circulating H₂S Blood was collected from 20 healthy volunteers at baseline, and again after one week of chronic exposure to a cocktail of oral, poorly-absorbed, broad-spectrum antibiotics. The mean concentration of total H₂S was calculated before and after antibiotics were administered. The difference between the mean values was calculated as percentage of pre-antibiotic levels.

Statistical analyses The Wilcoxon rank sum test or Mann-Whitney test for continuous variables were used to examine the difference between the groups where appropriate. Kruskal-Wallis test was used to compare analyte levels across age groups. All analyses were performed with RStudio-R version 4.1.2. (2021-11-01) (Vienna, Austria), or GraphPad Prism (version 9.1.2; GraphPad Software, Inc), and P-value<0.05 was considered statistically significant.

### Example 1

### Analytical Method Development

Internal standard synthesis and LC-MS/MS assay method development To serve as internal standard, [³⁴S]Na₂S was synthesized by reacting heavy isotope-labeled elemental sulfur [³⁴S] with metallic sodium in tetrahydrofuran under an anhydrous nitrogen atmosphere. Reaction conditions for an ethyl iodoacetate derivatization strategy were selected for analysis of sulfide (m/z 207.2), as described above (FIG. 1, Table 2). Analysis of the isotopic purity of the synthetic internal standard, [³⁴S]Na₂S (post-derivatization m/z 209.06454) showed 99.60±0.01% [³⁴S] enrichment (FIG. 1C), and its purity was confirmed by iodometric titration to be 108±1.4%. The ethyl iodoacetate derivatives of sulfide and [³⁴S]-sulfide were monitored using electrospray ionization in positive-ion mode, and three product ions for each isotopologue were selected for quantification using individual tuning (m/z 207.2→77.0, 207.2→105.0, 207.2→133.1 and m/z 209.2→79.1, 209.2→107.1, 209.2→135.1 for [³²S] and [³⁴S] derivatives, respectively). FIG. 2 shows the CID spectra (right) and co-chromatography (left) of selected multiple reaction monitoring (MRM) transitions for the quantification of natural-abundance and heavy isotope-labeled sulfide, using reverse phase liquid chromatographic conditions developed to retain and separate the ethyl iodoacetate derivatives of sulfide and other abundant plasma thiols (cysteine, homocysteine, glutathione, glutamylcysteine, cysteinylglycine). FIGS. 7 and 8 show the CID spectra and MRM transitions selected for natural abundance and heavy isotope enriched internal standards for these thiols. For each analyte monitored, at least two distinct precursor-product ion transitions were selected, and the specificity of the mass selection and fragmentation of the plasma thiols monitored simultaneously with sulfide gave the necessary compound specificity.

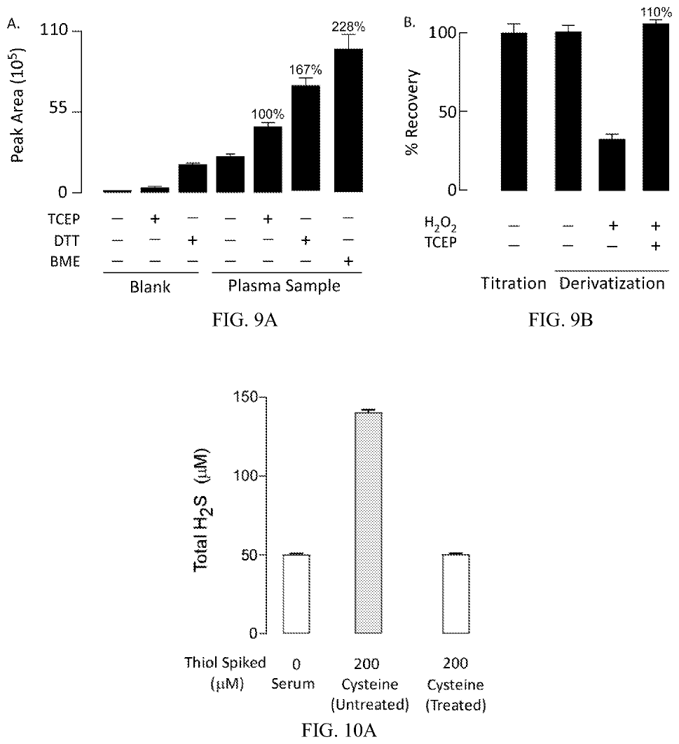

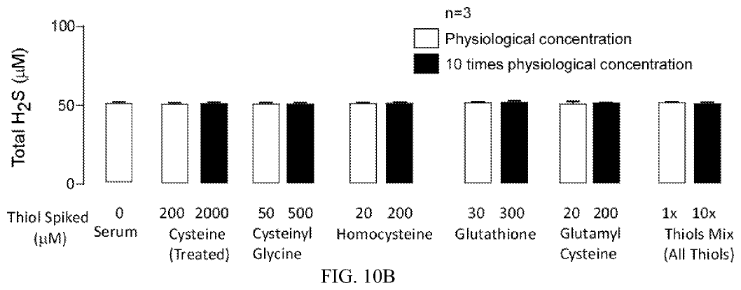

Selection of polysulfide reducing reagent and prevention of contaminant sulfide introduction during sample preparation for H₂S quantification Endogenous oxidants and post-collection oxidation processes can convert sulfide into polysulfide and mixed disulfides. To avoid H₂S oxidation, as well as H₂S production by iron and vitamin B6, diethylenetriaminepentaacetic acid (DTPA) was added to solutions during sample preparation. H₂S content artificially increased when using commercially acquired reductants like β-mercaptoethanol (BME) or dithiothreitol (DTT) at levels needed to quantitatively reduce polysulfides. This was avoided, as described above, and polysulfides were shown to be quantitatively recovered as free reduced H₂S using tris(2-carboxyethyl) phosphine (TCEP) as reductant (FIG. 9). Similarly, during spike and recovery studies with thiols monitored within the panel, significant artificial elevation of H₂S was observed when using commercial sources of thiols at physiological concentrations. After developing protocols to remove sulfide impurities, the assay methods developed showed quantitative recovery of each analyte within the panel without interference or artificial H₂S elevation, even when spiked with thiols at levels 10 times higher than physiological concentrations (FIG. 10).

Validation of the stable isotope dilution LC-MS/MS method for the quantitation of Total H₂S and thiols The H₂S quantification method was validated according to Clinical Laboratory Improvements and Amendments (CLIA) standards, included stability evaluation, and evaluation of matrix effect, linearity, lower limit of quantification (LLOQ), lower limit of detection (LLOD), accuracy, inter-day and intra-day imprecision, and substance interference. Pilot studies to determine storage conditions to prevent loss of the synthesized isotope-labelled internal standard (IS) (and verification at 2 months and then again after 1 year) resulted in storage of IS in airtight vials with minimal headspace and at −80° C. Under these conditions, the IS ([³⁴S]NA₂S) showed 93.9-3.9% recovery at 1 year (Table 3). The recovery of IS for H₂S and other monitored thiols was examined in human plasma by comparing analyte peak areas in pooled human serum versus vehicle. The percentage recovery of [³⁴S]-sodium sulfide, [3,3-D₂]-cysteine, [3,3,4,4,-D₄]-homocysteine and [¹³C₂,¹⁵N]-glutathione were all >96.8% (Table 4).

Stability The stability of H₂S and thiols in samples was determined through an accelerated stability study of pooled serum samples (Quality Control (QC) samples at 3 concentration levels) analyzed in triplicates at 60° C. for 5 days and compared with fresh matrix that was immediately processed. Sample stability was 94.4% for H₂S and 90-100.5% for the thiols in the panel. Freeze-thaw stability after 5 freeze-thaw cycles was 98.6% for H₂S and 90.9-110% for the thiols monitored. Long-term stability (1 year) was excellent, showing <10% bias for all analytes in the panel monitored (Table 5). Extract stability was also tested by reanalyzing QC extracts after ≥3 days at 10° C. (auto-sampler temperature) and quantifying against a freshly analyzed sample. The mean percent biases from the initial QC values for H₂S and thiol analytes in the panel were between-3.0% and 10.0%.

Matrix effect, linearity, LLOD and LLOQ After clearing commercial thiols of residual H₂S, the spike-and-recovery approach was used to generate calibration curves for H₂S and each thiol in the LC-MS/MS panel at eight different plasma concentrations. Comparing calibration curves in multiple different matrices, the calibration curves for H₂S and thiols in water and serum displayed minimal matrix effect across all analytes, with linear responses (r²>0.994) over 6 non-zero point calibration curves spanning physiological analyte concentration ranges. The LLOD was determined to be the lowest concentration of analyte in the sample with a signal-to-noise ratio ≥3 (2 nM for H₂S), and the LLOQ was determined to be the lowest concentration of analyte in the sample with a signal-to-noise ratio ≥10 (6 nM H₂S). LLOD and LLOQ for other thiols monitored in the panel are provided in Table 6.

Accuracy, and intra-day/inter-day imprecision Accuracy of the LC-MS/MS method was determined by comparing calculated versus measured analyte concentrations in 3 different serum pools spanning physiological concentrations (QC1 not spiked, QC2 and QC3 spiked with increasing amount of analyte). For H₂S, accuracy ranged 98.9-104.4%, and comparable accuracy was noted for each of the other thiols monitored in the panel (Table 1). Intra-day and inter-day imprecision of analyte values in serum were also evaluated at 3 QC levels (low, middle, high), and are shown in Table 1.

Interference and selectivity Interference studies of the effect of bilirubin, lipids (triglycerides) and hemolysis on the quantitation of H₂S and other thiols within the panel were performed on samples following procedures recommended by the Clinical and Laboratory Standards Institute. Briefly, icteric, lipemic, and hemolytic interferences were assessed using 6 concentrations of bilirubin (31.25-1000 μM), a triglyceride mix (0.375-12 mM), and hemolyzed red blood cells (0.25%-8%) spiked into three samples. Interferences with H₂S quantification showed biases of <2%. Bilirubin did not show significant interference in the quantification of any of the thiols. However, hemolytic interference with the quantification of cysteine, homocysteine, and glutathione were more pronounced (18.5-23.5%, 22.5-26.5%, and 33-9% respectively). Lipemic interference was detected only in glutathione, showing a bias of 15.8-44% when the concentration was increased in a dose-dependent manner, which presumably occurred by the release of glutathione from hemolyzed (rich in glutathione) red blood cell.

Comparison of collection tube type and carryover The impact of collection tube interference and differences was examined in blood sample types. Blood was collected from 10 donors into each of the following tube types: EDTA plasma, lithium heparin plasma, sodium citrate, and serum separator tubes. The samples were processed by standard procedures and the resulting paired plasma and serum samples from each donor were compared for differences in overall analyte quantification. No differences among tube types for all analytes monitored in the panel were noted, with sample differences ranging from −0.1% to 1.1%. Analyte carryover, determined by injecting water immediately after the highest standard, showed no detectable carryover for H₂S or any of the thiol analytes monitored.

TABLE 1
Determination of accuracy, precision, and recovery of the stable isotope dilution
LC-MS/MS method for quantification of Total H₂S and thiols in plasma.

| | | SAM | Mean | | | |
|---|---|---|---|---|---|---|
| | | concentration | concentration | Accuracy^{†} | Imprecision (CV %) | |
| Compound | | (μM) | (μM) | (%) | Intra-day^{‡} | Inter-day* |
| Hydrogen Sulfide | QC1 | 9.4 | 9.0 ± 1.1 | 104.4 | 7.6 | 12.7 |
| | QC2 | 19.5 | 21.8 ± 2.2 | 89.4 | 5.6 | 9.9 |
| | QC3 | 28.5 | 28.8 ± 1.6 | 98.9 | 4.0 | 5.7 |
| Cysteine | QC1 | 69.6 | 72.6 ± 6.7 | 95.9 | 8.3 | 9.2 |
| | QC2 | 267 | 254.2 ± 25.1 | 104.9 | 4.3 | 9.9 |
| | QC3 | 706 | 691.9 ± 25.6 | 102.0 | 1.3 | 3.7 |
| Homocysteine | QC1 | 7.2 | 6.2 ± 0.5 | 116.9 | 5.8 | 8.0 |
| | QC2 | 11.1 | 11.2 ± 0.9 | 98.6 | 4.5 | 7.6 |
| | QC3 | 45.3 | 45.5 ± 2.1 | 99.5 | 8.1 | 4.7 |
| Cysteinylglycine | QC1 | 6.2 | 5.8 ± 0.6 | 108.2 | 15.8 | 9.3 |
| | QC2 | 10.1 | 9.9 ± 1.0 | 101.2 | 4.2 | 11.8 |
| | QC3 | 22.1 | 22.3 ± 2.6 | 99.2 | 5.7 | 12.2 |
| Glutathione | QC1 | 3.7 | 3.3 ± 0.3 | 109.6 | 7.5 | 9.7 |
| | QC2 | 9.2 | 9.5 ± 1.1 | 96.7 | 6.9 | 10.5 |
| | QC3 | 24.0 | 23.5 ± 2.9 | 101.9 | 5.2 | 11.5 |
| Glutamylcysteine | QC1 | 0.8 | 0.8 ± 0.1 | 102.1 | 2.3 | 11.5 |
| | QC2 | 2.6 | 2.7 ± 0.4 | 94.4 | 5.7 | 14.4 |
| | QC3 | 6.6 | 6.7 ± 0.8 | 99.0 | 7.0 | 11.8 |

^{‡}Inter-day Imprecision (CV, %) = 100 × (standard deviation/mean concentration).
^{†}Accuracy (%) = 100 × mean concentration/SAM concentration.
*Intraday Imprecision (CV, %) = 100 × standard deviation/mean concentration.

TABLE 2
Selection of the reagent for derivatizing H₂S and thiols.

| Reagent | Peak Area | Peak Area [%] |
|---|---|---|
| Ethyl iodoacetate | 6.0 × 10⁸ | 100 |
| Ethyl bromoacetate | 4.8 × 10⁸ | 80 |
| Methyl bromoacetate | 8.5 × 10⁵ | 0.1 |
| Iodoacetate | 6.0 × 10⁷ | 10 |

The H₂S derivatized with each reagent was analyzed by LC-MS/MS and the yield of each derivatization reaction (relative to ethyl iodoacetate) was estimated from the peak area of the MS1 ion in each chromatogram.

TABLE 3
Determination of the stability of the synthesized
isotope labelled internal standard [³⁴S]Na₂S.

| Storage at −80° C. | Peak Area × 10⁶ | % |
|---|---|---|
| 1 Week | 1.22 ± 0.12 | 100.0 ± 9.7 |
| 2 Week | 1.23 ± 0.10 | 101.3 ± 8.5 |
| 3 Week | 1.18 ± 0.17 | 97.3 ± 14.3 |
| 4 Week | 1.23 ± 0.10 | 101.1 ± 8.1 |
| 5 Week | 1.17 ± 0.05 | 96.1 ± 4.5 |
| 6 Week | 1.23 ± 0.10 | 101.1 ± 8.1 |
| 7 Week | 1.26 ± 0.16 | 103.7 ± 12.3 |
| 8 Week | 1.24 ± 0.26 | 98.8 ± 19.9 |
| 1 year | 1.14 ± 0.04 | 93.9 ± 3.9 |

TABLE 4
Recovery of H₂S/thiols heavy isotope labelled internal
standards spiked in human plasma versus water.

| | Mean area of | Mean area of | |
|---|---|---|---|
| | analyte spiked | analyte spiked | Recovery^{‡} |
| Compound | in serum | in water | (%) |
| [³⁴S]-Sodium | (1.16 ± 0.07) × 10⁶ | (1.11 ± 0.17) × 10⁶ | 104.6 |
| sulfide | | | |
| [3,3-D₂]-Cysteine | (6.13 ± 0.05) × 10⁵ | (6.27 ± 0.05) × 10⁵ | 97.9 |
| [3,3,4,4,-D₄]- | (1.22 ± 0.08) × 10⁵ | (1.19 ± 0.11) × 10⁵ | 102.8 |
| Homocysteine | | | |
| [¹³C2,¹⁵N]- | (1.07 ± 0.06) × 10⁵ | (1.11 ± 0.07) × 10⁵ | 96.8 |
| Glutathione | | | |

^{‡}The recovery of spiked heavy isotope labelled H₂S and thiols in human plasma was calculated according to the following formula: Recovery (%) = 100 × Mean area of analyte spiked in serum/Mean area of analyte spiked in water.

TABLE 5
Long term stability study of H₂S and
thiols in human plasma samples (n = 3)

| | | Initial mean | Mean | |
|---|---|---|---|---|
| | | concentration, | concentration, | |
| | | C₀ | Cₜ | Stability^{‡} |
| Compound | | (μM) | (μM) | (%) |
| H₂S | QC1 | 9.7 ±0.03 | 9.6 ±0.2 | 98.4 ±2.2 |
| | QC2 | 24.3 ± 0.6 | 24.5 ± 1.0 | 97.4 ± 4.3 |
| | QC3 | 29.4 ± 1.5 | 29.5 ± 0.6 | 99.0 ± 3.9 |
| Cysteine | QC1 | 74.9 ± 7.4 | 75.0 ± 2.3 | 96.9 ± 6.0 |
| | QC2 | 256.4 ± 6.9 | 247.0 ± 7.5 | 96.6 ± 3.2 |
| | QC3 | 700.6 ± 3.7 | 693.9 ± 5.9 | 100.5 ± 1.1 |
| Homocysteine | QC1 | 6.5 ± 0.5 | 5.2 ± 0.3 | 90.3 ± 5.6 |
| | QC2 | 11.2 ± 0. | 11.6 ± 0.6 | 101.3 ± 5.3 |
| | QC3 | 43.8 ± 2.7 | 50.3 ± 3.5 | 108.2 ± 6.9 |
| Glutathione | QC1 | 2.7 ± 0.1 | 2.3 ± 0.3 | 82.1 ± 4.9 |
| | QC2 | 10.9 ± 0.5 | 10.7 ± 0.3 | 95.8 ± 5.5 |
| | QC3 | 26.4 ± 0.4 | 26.8 ± 1.9 | 99.0 ± 6.1 |
| Glutamylcysteine | QC1 | 0.8 ± 0.03 | 0.7 ± 0.01 | 95.6 ± 1.3 |
| | QC2 | 3.6 ± 0.4 | 3.1 ± 0.1 | 96.2 ± 3.5 |
| | QC3 | 7.1 ± 0.4 | 7.1 ± .0.6 | 94.3 ± 6.9 |
| Cysteinylglycine | QC1 | 5.6 ± 0.5 | 4.8 ± 0.3 | 91.9 ± 6.5 |
| | QC2 | 10.6 ± 0.1 | 10.9 ± 0.3 | 99.0 ± 3.5 |
| | QC3 | 23.9 ± 0.1 | 22.2 ± 0.8 | 94.4 ± 4.9 |

^{‡}Stability was calculated with the following formula: % Stability = 100 × Cₜ/C₀, where C₀ is the initial mean concentration of Total H₂S/thiols in the fresh serum sample, and Cₜ is the mean concentration after one year storage.

TABLE 6
Determination of lower limits of quantitation (LLOQ) and detection
(LLOD) of H₂S and thiols in human plasma samples.

| Compound | LLOQ^{†} (nM) | LLOD^{‡} (nM) |
|---|---|---|
| Hydrogen Sulfide | 6 | 2 |
| Cysteine | 170 | 51 |
| Homocysteine | 234 | 70 |
| Cysteinylglycine | 55 | 17 |
| Glutathione | 833 | 250 |
| Glutamylcysteine | 78 | 23 |

^{‡}The LLOD was determined to be the lowest concentration of analyte in the sample with a signal-to-noise ratio higher than 3.
^{†}The LLOQ was determined to be the lowest concentration of analyte in the sample with a signal-to-noise ratio higher than 10.

### Example 2

### Clinical Uses

Establishing reference ranges for circulating Total H₂S and thiols monitored in healthy volunteers Reference ranges of total H₂S and thiols in plasma were determined using samples collected from apparently healthy subjects (n=200) recruited from community health screenings. Subjects were considered healthy if they reported no medical history of cardiovascular disease, history, or laboratory findings consistent with diabetes, cardiometabolic disorders, or cancer, no reported use of any medications, and showed normal laboratory screening results for basic metabolic and lipid panels (Table 7). The normal range of plasma concentrations was determined to be 4.2-62.7 μM for total H₂S, 46.1-463.8 UM for total cysteine, 1.5-16.5 μM for total homocysteine, 3.1-90.3 μM for total cysteinylglycine, 0.8-16.9 μM for total glutamylcysteine, and 0.3-12.0 μM for total glutathione (Table 8).

Clinical cohort study population Participants (n=400) were stable consenting subjects undergoing general health or risk factor evaluation. The mean age in the cohort was 65.2±11.0 years, 50% were men, 94.8% were Caucasian, and the mean BMI was 29.0±5.6 kg/m². Median (interquartile range) of total cholesterol was 164 (138.4-191.1) mg/dL, triglycerides was 112.0 (85.0-168.0) mg/dL, HDL cholesterol was 37.9 (30.4-48.1) mg/dL and LDL cholesterol was 95.0 (74.0-119.5) mg/dL.

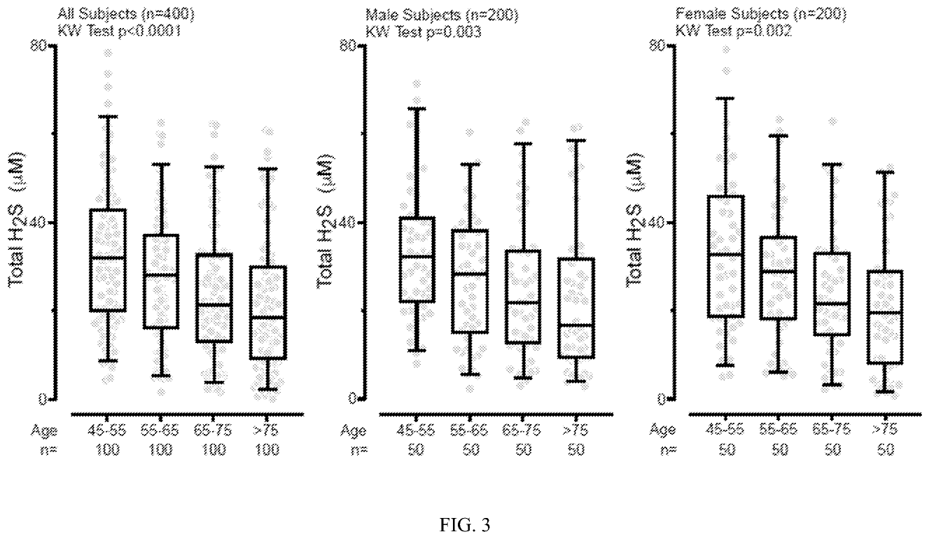

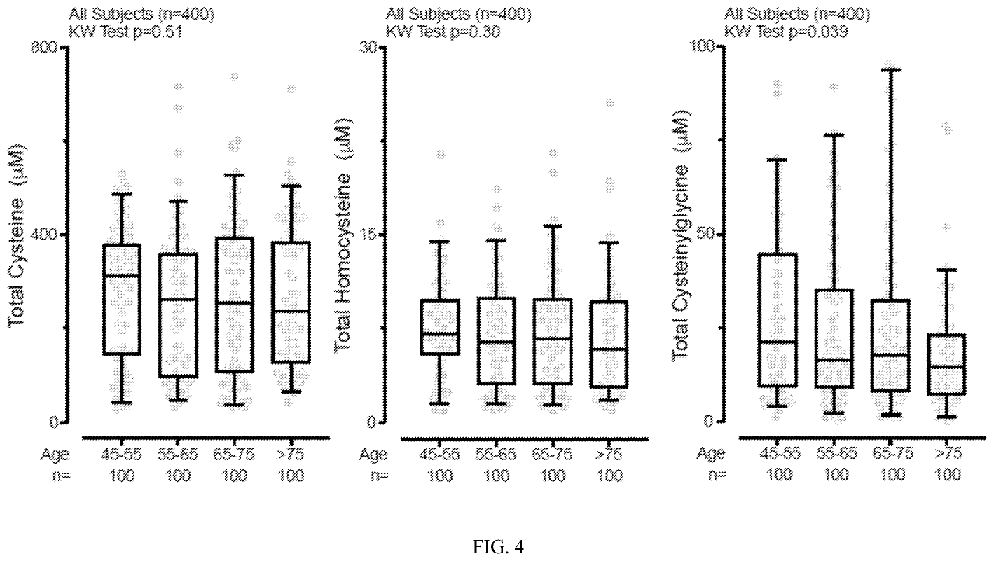

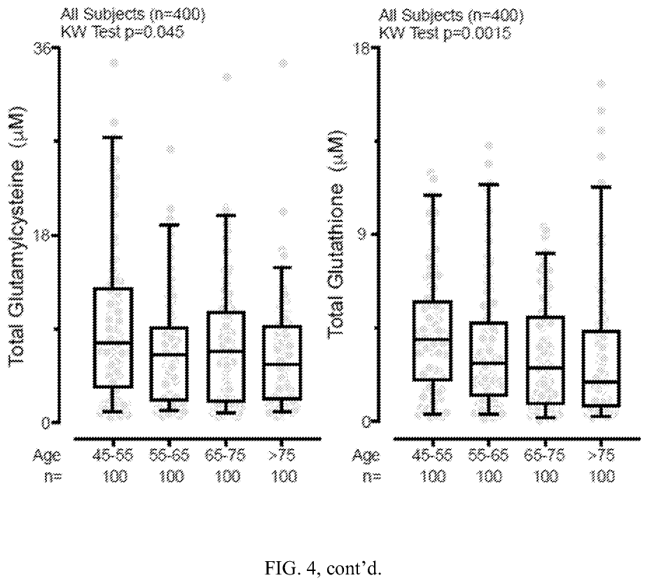

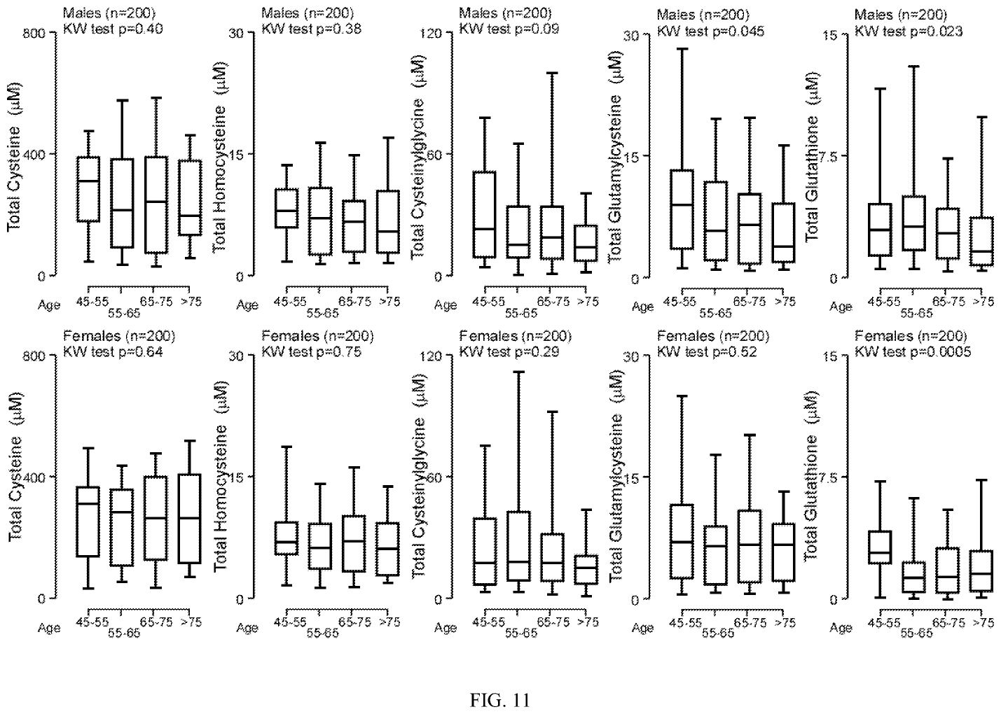

Plasma levels of Total H₂S decrease with age Numerous studies suggest that production of H₂S and its downstream protein persulfidation diminishes with age. The method developed herein was used to determine plasma levels of total H₂S and thiols in an independent clinical cohort (n=400) to examine the association of H₂S with aging. The analyses shown in FIG. 3 reveal that total H₂S levels decrease with age (p<0.0001). This trend was observed in both males (p=0.003) and females (p=0.002) (FIG. 3). Age-associated reductions in plasma concentrations of total cysteinylglycine, total glytamylcysteine, and total glutathione (FIG. 4) were also observed in the combined male/female cohort, but when data were separated by gender, only males had a statistically significant reduction across age categories examined in total glutamylcysteine levels (p=0.045), while both males and females showed significant reduction in total glutathione levels (males p=0.023, females p=0.0005), FIG. 11).

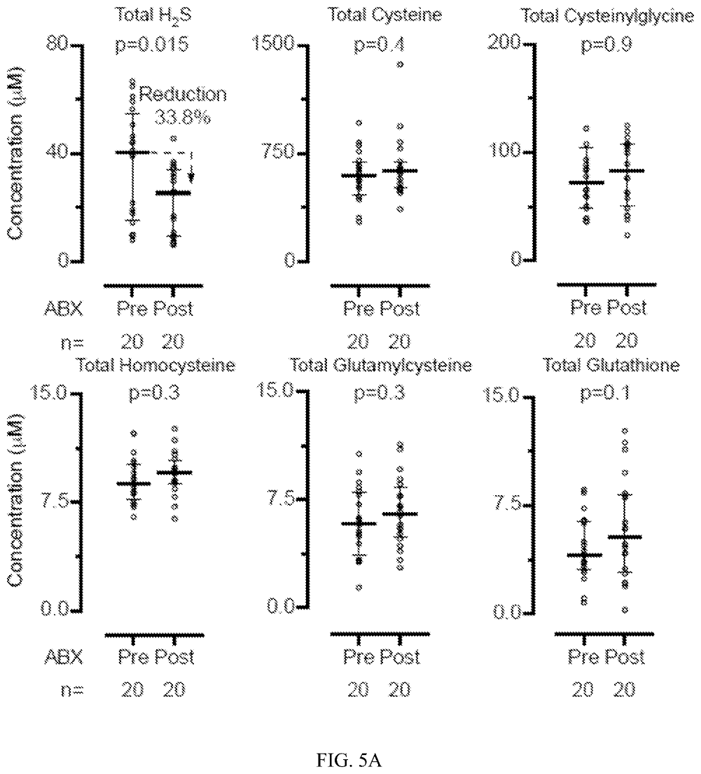

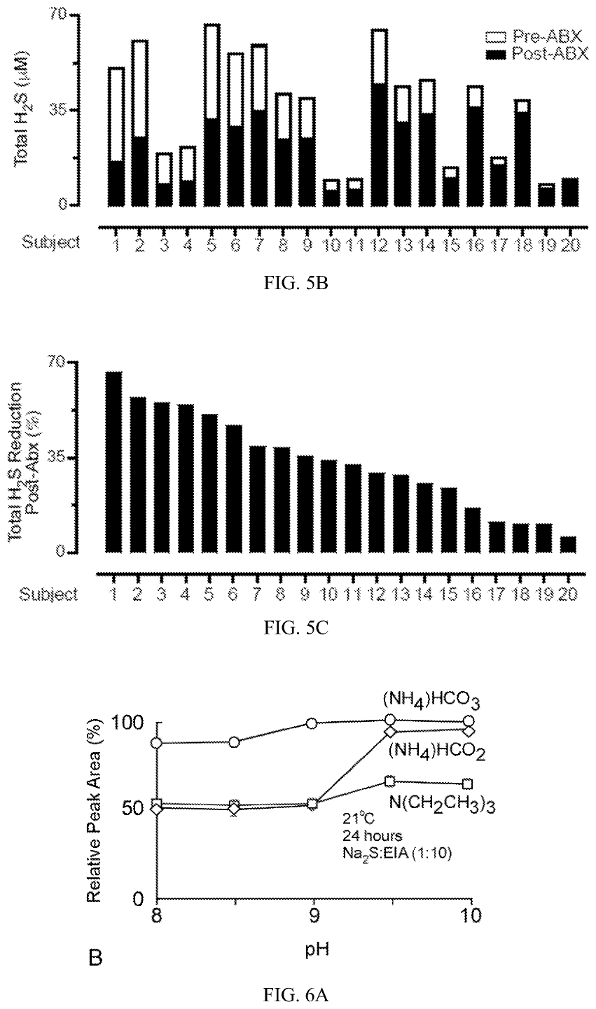

Gut microbiota contributes significantly to circulating levels of Total H₂S in humans While it is widely recognized that some gut microbes can produce H₂S, and one study reports that germ-free mice have decreased plasma H₂S levels, the contribution of gut microbiota to systemic H₂S levels in humans is unknown. Circulating levels of total H₂S and thiols were quantified in a cohort of 20 healthy volunteers before and after ≥5 days of an oral cocktail of poorly-absorbed broad-spectrum antibiotics previously shown to suppress gut microbiota. On average, total H₂S concentrations were diminished by 33.8±17.7% (p=0.015, Mann-Whitney test) following suppression of gut microbiota (FIG. 5A). Post antibiotics treatment a 29.2% reduction of total H₂S levels was observed in females and 37.6% was observed in males, however there was no significant differences in total H₂S reduction between males and females (p=0.94, Mann-Whitney test). In addition, none of the other thiols monitored showed reduction following gut microbiota suppression (FIG. 5A). Notably, the contributions of gut microbiota to systemic total H₂S levels varied widely across subjects (FIG. 5B), ranging from only 6.0% to 66.7% (FIG. 5C). For example, in 25% of the subjects (subjects 1-5, FIG. 5C), >50% reduction in circulating total H₂S was observed post-antibiotics exposure, suggesting the majority of systemic H₂S originated from gut microbiota source in these subjects. In contrast, 15% of the subjects (subjects 17-20) showed <10% reduction following antibiotics.

TABLE 7
Clinical characteristics of the participants in the cohort used
to establish normal range for Total H₂S and thiols in human
plasma through the stable isotope dilution LC-MS/MS method.

| | Healthy |
|---|---|
| | subjects |
| Characteristic | (n = 200) |
| Age (years) | 59.9 ±11.8 |
| Male (%) | 31.1 |
| Caucasians (%) | 95.7 |
| BMI (kg/m²) | 27.5 ± 5.7 |
| Total cholesterol (mg/dL) | 172.9 ± 25.3 |
| HDL (mg/dL) | 55.3 ± 14.2 |
| LDLc (mg/dL) | 95.8 ± 20.9 |
| Triglycerides (mg/dL) | 86.1 ± 35.8 |
| Diabetes | 0 |
| CVD | 0 |
| CKD | 0 |

TABLE 8
Clinical characteristics of the participants in the cohort used
to establish normal range for Total H₂S and thiols in human
plasma through the stable isotope dilution LC-MS/MS method.

| | | | 95% | |
|---|---|---|---|---|
| | | | Normal | |
| | Mean | 2.5^{th} | range | 97.5^{th} |
| Compound | (μM) | Percentile | (μM) | Percentile |
| Hydrogen Sulfide | 26.7 ± 15.6 | 4.76 | 4.2-62.7 | 61.93 |
| Cysteine | 228.4 ± 126.2 | 63.19 | 46.1-463.8 | 459.4 |
| Homocysteine | 6.6 ± 4.2 | 1.64 | 1.5-16.5 | 14.66 |
| Cysteinylglycine | 17.3 ± 12.9 | 3.44 | 3.1-90.3 | 85.77 |
| Glutamylcysteine | 5.7 ± 4.8 | 0.82 | 0.8-16.9 | 16.37 |
| Glutathione | 3.15 ± 2.9 | 0.28 | 0.3-12 | 11.7 |

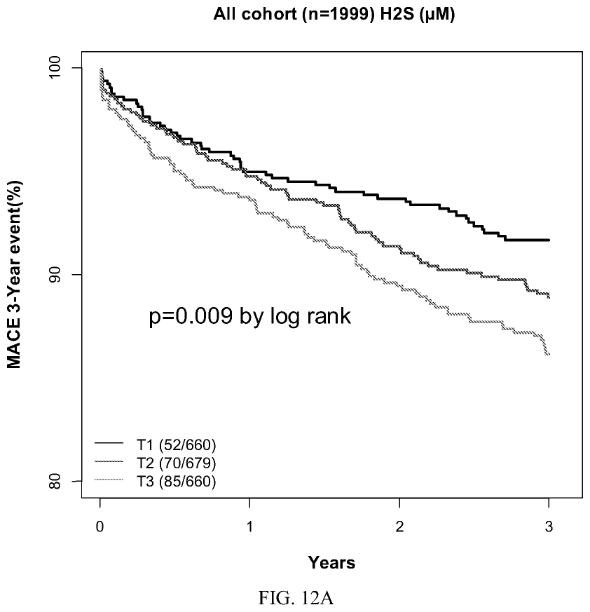

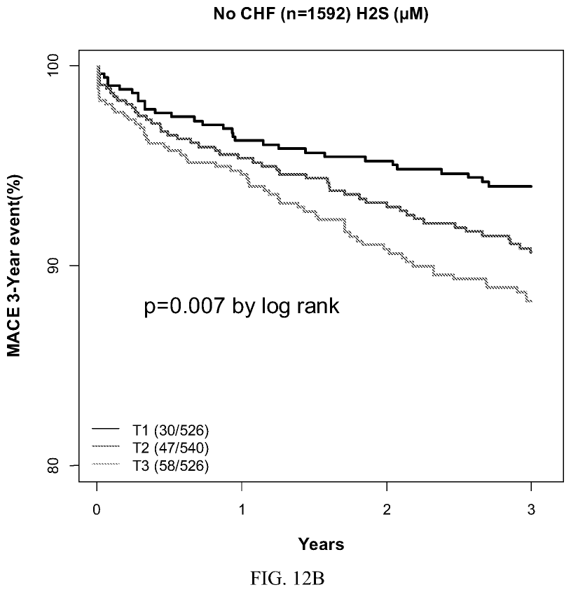

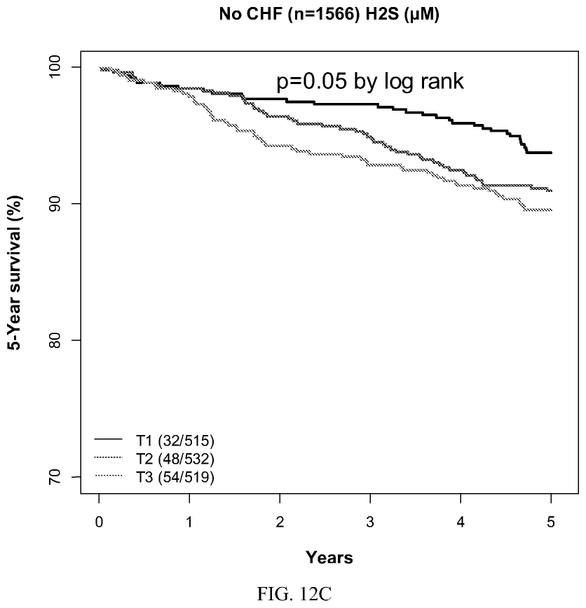

Subjects total H₂S levels was determined in a cohort of sequential subjects at baseline using the disclosed LC/MS/MS method, and the relationship with incident major adverse cardiac events (MACE=myocardial infarction (MI), stroke, or death) over the ensuing 3 year period was determined. Shown in FIG. 12A is the Kaplan Meier plot analyses of increasing tertile levels of total H₂S versus freedom from MACE event risk in the entire cohort. In this study cohort, which consists of sequential subjects undergoing cardiac risk assessment, a higher total H₂S level predicted increased incident risk for MACE in a dose dependent manner.

Total H₂S levels were determined at baseline among the indicated number of sequential subjects without congestive heart failure using the disclosed LC/MS/MS method, and the relationship with incident major adverse cardiac events over the ensuing 3 year period was determined. Shown in FIG. 12B is the Kaplan Meier plot analyses of increasing tertile levels of total H₂S versus freedom from MACE event risk. In this study cohort, which consists of sequential nonCHF subjects undergoing cardiac risk assessment, a higher total H₂S level predicted increased incident risk for MACE (MI, stroke or death) in a dose dependent manner.

Subjects total H₂S levels was determined at baseline using the disclosed LC/MS/MS method, and the relationship with incident major adverse cardiac events over the ensuing 5 year period was determined. Shown in FIG. 12C is the Kaplan Meier plot analyses of increasing tertile levels of total H₂S versus freedom from all-cause mortality in the entire cohort. In this study cohort, which consists of sequential subjects undergoing cardiac risk assessment, a higher total H₂S level predicted increased incident mortality risk in a dose dependent manner.

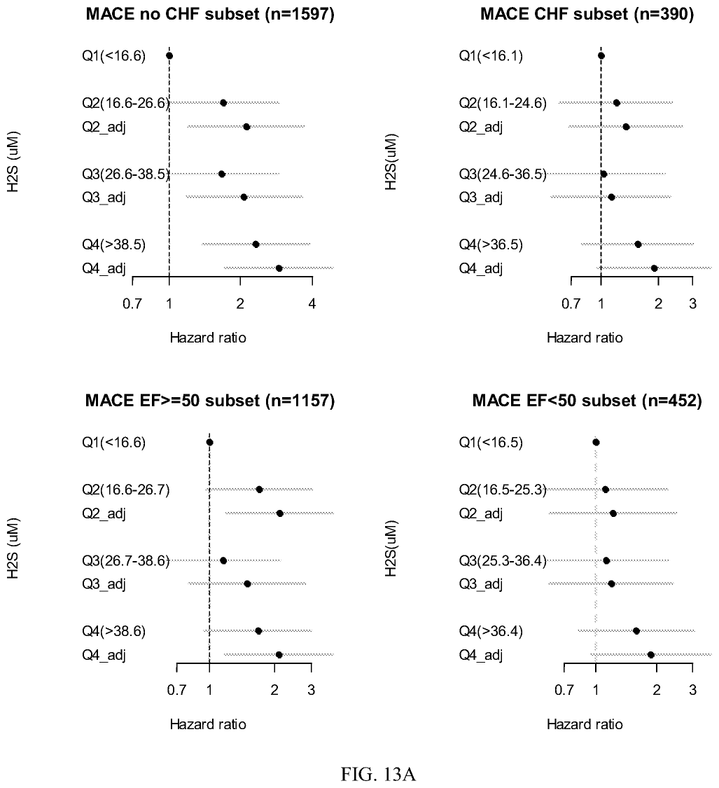

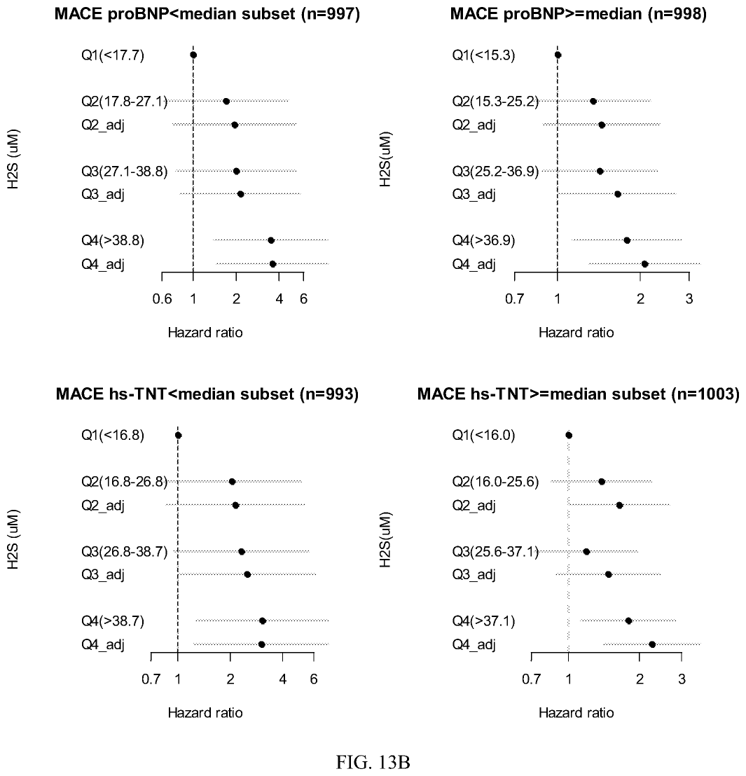

Subjects total H₂S levels was determined at baseline using the disclosed LC/MS/MS method, and the relationship with incident major adverse cardiac events (MACE=MI, stroke or death) over the ensuing 3 year period in the indicated subset of subjects was determined. In all subsets examined (FIG. 13A), a higher total H₂S level was associated with tendency toward higher hazard ratio for incident risk for MACE in a dose dependent manner-even amongst subjects with no evidence of heart failure, or normal cardiac function.

Subjects total H₂S levels was determined at baseline using the disclosed LC/MS/MS method, and the relationship with incident major adverse cardiac events over the ensuing 3 year period in the indicated subset of subjects was determined. In all subsets examined (FIG. 13B), a higher total H₂S level was associated with tendency toward higher hazard ratio for incident risk for MACE in a dose dependent manner-even amongst subjects with and without evidence of subclinical myocardial necrosis (e.g., elevated versus normal high sensitivity TnT level) or with and without evidence of subclinical myocardial strain (e.g., elevated versus normal NTproBNP level).

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

What is claimed is:

1. A method for detecting hydrogen sulfide in a sample comprising:
adding a ³⁴S isotope-labeled sulfide compound and a reducing agent to the sample;
incubating the sample with a derivatizing agent to form a derivatized sample; and
analyzing the derivatized sample by isotope dilution mass spectrometry.

2. The method of claim 1, further comprising:
detecting primary and secondary multiple reaction monitoring (MRM) transitions for ³⁴S isotope-labeled derivatized sulfide and unlabeled derivatized sulfide;
calculating an area ratio of the primary unlabeled derivatized sulfide MRM transition to the ³⁴S isotope-labeled derivatized sulfide primary MRM transition; and
determining a concentration of hydrogen sulfide by multiplying the concentration of added ³⁴S isotope-labeled sulfide compound with the area ratio.

3. The method of claim 1 or 2, wherein the ³⁴S isotope-labeled sulfide compound is ³⁴S isotope-labeled sodium sulfide.

4. The method of claim 3, wherein the ³⁴S isotope-labeled sodium sulfide has an isotopic abundance of greater than 90% ³⁴S.

5. The method of any of claims 1-4, wherein the ³⁴S isotope-labeled sulfide is added in a basic buffer solution.

6. The method of claim 5, wherein the basic buffer solution is ammonium bicarbonate at pH 10.

7. The method of any of claims 1-6, wherein the reducing agent is an agent capable of reducing polysulfides.

8. The method of any of claims 1-7, wherein the reducing agent is tris(2-carboxyethyl) phosphine (TCEP).

9. The method of any of claims 1-8, wherein the reducing agent is added to the sample at a final concentration of at least 1 mM.

10. The method of claim 9, wherein the final concentration of the reducing agent is 1-100 mM.

11. The method of any of claims 1-10, wherein the ³⁴S isotope-labeled sulfide compound and the reducing agent are added simultaneously.

12. The method of any of claims 1-11, further comprising adding a chelating agent to the sample.

13. The method of claim 12, wherein the chelating agent is diethylenetriaminepentaacetic acid (DTPA).

14. The method of any of claims 1-13, wherein the derivatizing agent is selected from ethyl iodoacetate, ethyl bromoacetate, methyl bromoacetate, and iodoacetate.

15. The method of any of claims 1-14, wherein the derivatizing agent is ethyl iodoacetate.

16. The method of any of claims 1-15, wherein the derivatizing agent is present in a molar excess compared to hydrogen sulfide in the sample.

17. The method of any of claims 1-16, wherein the derivatizing agent is at a molar ratio of hydrogen sulfide to derivatizing agent of at least about 1:10.

18. The method of any of claims 1-17, wherein the incubating is conducted for a time sufficient to allow detection of derivative products by mass spectrometry.

19. The method of any of claims 1-18, wherein the incubating is 8-24 hours.

20. The method of any of claims 1-19, wherein the incubating is conducted at a temperature sufficient to allow detection of derivative products by mass spectrometry.

21. The method of any of claims 1-20, wherein the incubating is at 20-23° C.

22. The method of any of claims 1-21, further comprising removing proteins in the derivatized sample.

23. The method of any of claims 1-22, wherein the isotope dilution mass spectrometry comprises liquid chromatography and tandem mass spectrometry.

24. The method of any of claims 2-23, wherein the primary MRM for unlabeled derivatized sulfide is 207.2→133.1 and the primary MRM for ³⁴S isotope-labeled derivatized sulfide is 209.2→135.1.

25. The method of any of claims 2-24, wherein the secondary MRM for unlabeled derivatized sulfide is 207.2→105.0 and the secondary MRM for ³⁴S isotope-labeled derivatized sulfide is 209.2→107.1.

26. The method of any of claims 2-25, further comprising calculating the area ratio of the unlabeled derivatized sulfide secondary MRM transition to the ³⁴S isotope-labeled derivatized sulfide secondary MRM transition and comparing to area ratio of primary MRM transitions.

27. The method of any of claims 1-26, further comprising detecting additional thiol species in the sample by isotope dilution mass spectrometry.

28. The method of any of claims 1-27, further comprising:
adding an isotope labeled thiol compound to the sample;

29. The method of claim 28, further comprising:
detecting primary and secondary MRM transitions for one or more or all of: cysteine, homocysteine, glutathione, cysteinylglycine and glutamylcysteine;
calculating an area ratio of the primary MRM transition for one or more or all of: cysteine, homocysteine, glutathione, cysteinylglycine and glutamylcysteine to a corresponding isotope labeled thiol compound primary MRM transition; and
determining a concentration by multiplying the concentration of corresponding isotope labeled thiol compound with the area ratio.

30. The method of claim 27 or 29, wherein the isotope labeled thiol compound comprises one or more or all of: [3,3-D₂] cysteine, [3,3,4,4-D₄] homocysteine, and (¹³C₂¹⁵N) glutathione.

31. The method of any one of claims 28-30, wherein the isotope labeled thiol compound is substantially free of H₂S.

32. The method of any of claims 1-31, wherein the sample is a biological sample from a subject.

33. The method of any of claims 1-32, wherein the biological sample is blood or a blood product.

34. The method of claim 31 or 32, further comprising predicting risk of a disease or disorder or death in a subject, comprising comparing the hydrogen sulfide concentration from a subject sample to a control, wherein an increased hydrogen sulfide concentration indicates an increased risk of the disease or disorder.

35. The method of claim 34, wherein the disease or disorder comprises cardiovascular disease.

36. The method of claim 35, wherein the cardiovascular disease is selected from angina, arrhythmia, arteriosclerosis, atherosclerosis, myocardial infarction, acute coronary syndrome, cardiomyopathy, congestive heart failure, coronary thrombosis, aortic aneurysm, aortic dissection, iliac or femoral aneurysm, pulmonary embolism, high blood pressure/hypertension (e.g., primary hypertension), hypercholesterolemia/hyperlipidemia, atrial fibrillation, stroke, transient ischemic attack, systolic dysfunction, diastolic dysfunction, myocarditis, atrial tachycardia, ventricular fibrillation, endocarditis, arteriopathy, vasculitis, atherosclerotic plaque, vulnerable plaque, acute ischemic attack, sudden cardiac death, peripheral vascular disease, coronary artery disease (CAD), carotid artery disease, peripheral artery disease (PAD), cerebrovascular disease, adverse ventricular remodeling, ventricular systolic dysfunction, ventricular diastolic dysfunction, cardiac dysfunction, ventricular arrhythmia, and stroke.

37. The method of claim 34, wherein the disease or disorder comprises a disease or disorder associated with aging.

38. The method of claim 37, wherein the disease or disorder is selected from impaired cognitive function, Alzheimer's Disease, arthritis, osteoporosis, macular degeneration, dementia, and Type 2 diabetes.

39. The method of claim 34, wherein the disease or disorder is selected from non-alcoholic steatohepatitis (NASH), kidney disease, adverse ventricular remodeling, ventricular systolic dysfunction, ventricular diastolic dysfunction, cardiac dysfunction, and ventricular arrhythmia.

40. The method of any one of claims 34-39, wherein, if the method identifies that the subject has a risk of a cardiovascular disease or disorder, the method further comprises treating the subject with at least one treatment for cardiovascular disease.

41. The method of claim 40, comprising implementing a treatment regimen selected from an adjusted dietary regimen, an exercise regimen, administering a cholesterol lowering agent, administering a blood pressure modifying agent, or any combination thereof.

42. The method of claim 40 or 41, comprising administering to the subject an agent selected from a statin, a fibrate, niacin, a bile acid resin, a cholesterol absorption inhibitor, a phytosterol, an alginate, a pectin, lecithin, or nutraceutical.

43. The method of any one of claims 40-42, comprising administering to the subject an agent selected from Omega 3 oil, salicylic acid, dimethylbutanol, garlic oil, olive oil, krill oil, Co enzyme Q-10, a probiotic, a prebiotic, dietary fiber, psyllium husk, bismuth salts, phytosterols, grape seed oil, green tea extract, vitamin D, antioxidants, turmeric, curcumin, and resveratrol.

44. A composition comprising sodium sulfide, wherein greater than 90% of the sodium sulfide is ³⁴S isotope-labeled sodium sulfide.

45. The composition of claim 44, wherein greater than 95% of the sodium sulfide is ³⁴S isotope-labeled sodium sulfide.

46. The composition of claim 44, wherein greater than 99% of the sodium sulfide is ³⁴S isotope-labeled sodium sulfide.

47. The composition of any of claims 44-46, wherein the composition further comprises a basic buffer solution.

48. The composition of claim 47, wherein the basic buffer solution comprises ammonium bicarbonate.

49. The composition of claim 47 or 48, wherein the basic buffer solution has a pH of 9-11.

50. The composition of any of claims 47-49, wherein the basic buffer solution is ammonium bicarbonate at pH 10.

51. A method of making ³⁴S isotope-labeled sodium sulfide comprising
mixing elemental sulfur (³⁴S) and metallic sodium at about a 1:2 molar ratio;
heating the mixture for greater than about 18 hours;
collecting resulting precipitate; and
drying ³⁴S isotope-labeled sodium sulfide solid.

52. The method of claim 51, wherein the ³⁴S isotope-labeled sodium sulfide solid has an isotopic abundance of greater than 90% ³⁴S.

53. The composition of claim 51, the ³⁴S isotope-labeled sodium sulfide solid has an isotopic abundance of greater than 95% ³⁴S.

54. The composition of claim 51, the ³⁴S isotope-labeled sodium sulfide solid has an isotopic abundance of greater than 99% ³⁴S.

55. A kit comprising the composition of any of claims 44-50 and at least one of: a reducing agent, a derivatization agent, a chelating agent, an isotopically-labeled thiol-containing compound, a buffer, a solvent, and a container.

56. The kit of claim 55, wherein the reducing agent is an agent capable of reducing polysulfides.

57. The kit of claim 55 or 56, wherein the reducing agent is TCEP.

58. The kit of any of claims 55-57, wherein the derivatizing agent is selected from ethyl iodoacetate, ethyl bromoacetate, methyl bromoacetate, iodoacetate, and combinations thereof.

59. The kit of any of claims 55-58, wherein the derivatizing agent is ethyl iodoacetate.

60. The kit of any of claims 55-59, wherein the chelating agent is diethylenetriaminepentaacetic acid (DTPA).

61. The kit of any of claims 55-60, wherein the isotopically-labeled thiol-containing compound comprises [3,3-D₂] cysteine, [3,3,4,4-D₄] homocysteine, (¹³C₂¹⁵N) glutathione, or a combination thereof.

62. The kit of any of claims 55-59, wherein the kit comprises a sealable reaction vial.

63. The kit of any of claims 55-62, wherein the solvent is substantially free of sulfur-containing compounds.

64. Use of an ³⁴S isotope-labeled sulfide compound to detect hydrogen sulfide in a sample.

65. The use of claim 64, wherein the ³⁴S isotope-labeled sulfide compound is ³⁴S isotope-labeled sodium sulfide.

66. The use of claim 65, wherein the ³⁴S isotope-labeled sodium sulfide has an isotopic abundance of greater than 90% ³⁴S.

67. The use of claim 66, wherein the ³⁴S isotope-labeled sodium sulfide has an isotopic abundance of greater than 95% ³⁴S.

68. The use of claim 67, wherein the ³⁴S isotope-labeled sodium sulfide has an isotopic abundance of greater than 99% ³⁴S.

69. The use of any of claims 64-68, wherein the sample is a biological sample from a subject.

70. The use of claim 69, wherein the biological sample is blood or a blood product.
